(19) United States
(12) Patent Application Publication
(10) Pub. No.: US 2022/0218751 A1
(43) Pub. Date: Jul. 14, 2022

(54) ENHANCEMENT OF POLYPEPTIDES AND CHIMERIC ANTIGEN RECEPTORS VIA HINGE DOMAINS

(71) Applicant: The Board of Trustees of the Leland Stanford Junior University, Stanford, CA (US)

(72) Inventors: Robbie G. Majzner, Palo Alto, CA (US); Crystal L. Mackall, Stanford, CA (US); Louai Labanieh, Palo Alto, CA (US); Skyler P. Rietberg, San Francisco, CA (US)

(21) Appl. No.: 17/608,709

(22) PCT Filed: May 6, 2020
(86) PCT No.: PCT/US2020/031728

§ 371 Date: Nov. 3, 2021

Related U.S. Application Data
(60) Provisional application No. 62/844,683, filed on May 7, 2019.

(51) Int. Cl. A61K 35/17; C07K 14/705; C07K 14/725; C07K 14/715; C07K 14/73; A61K 35/76; A61P 35/00

(52) U.S. Cl. CPC A61K 35/17; C07K 14/70521; C07K 14/7051; C07K 14/7151; A61K 38/00; C07K 14/70517; C07K 14/70514; A61K 35/76; A61P 35/00; C07K 14/70507

(57) ABSTRACT

The present disclosure generally relates to, inter alia, novel chimeric polypeptides and chimeric antigen receptors (CARs) that include a hinge domain from CD28 and optionally a costimulatory domain not from CD28. The disclosure also provides compositions and methods useful for producing such molecules, as well as methods for the detection and treatment of diseases, such as cancer.

### CROSS-REFERENCE TO RELATED APPLICATION

This application claims the benefit of priority to U.S. Provisional Patent Application Ser. No. 62/844,683, filed on May 7, 2019. The disclosure of the above-referenced application is herein expressly incorporated by reference it its entirety, including any drawings.

### STATEMENT REGARDING FEDERALLY SPONSORED R&D

The invention was made with government support under grant no. 1P01CA217959 awarded by the National Institutes of Health grant no. U54 CA232568-01 awarded by the National Cancer Institute. The government has certain rights in the present invention.

### INCORPORATION OF THE SEQUENCE LISTING

The material in the accompanying Sequence Listing is hereby incorporated by reference into this application. The accompanying Sequence Listing text file, named 078430-506001WO-Sequence Listing.txt, was created on Apr. 20, 2020 and is 80 KB.

### FIELD

The present disclosure relates generally to the fields of oncology and immuno-therapeutics, and particularly relates to novel polypeptides, e.g., chimeric antigen receptors that include a hinge domain from CD28 and optionally a costimulatory domain not from CD28. The disclosure also provides compositions and methods useful for producing such molecules, as well as methods for the detection and treatment of conditions, such as diseases (e.g., cancer).

### BACKGROUND

In recent years, chimeric antigen receptors (CARs) have emerged as a promising approach for immunotherapy and made headlines in clinical trials conducted by a number of pharmaceutical and biotechnology companies. CARs are antigen-specific recombinant receptors, which, in a single molecule, redirect the specificity and function of a number of immune cells, including T lymphocytes, natural killer (NK) cells, natural killer T (NKT) cells, and macrophages. For example, in CAR-T cell therapy, the general premise for the use of CAR-T cells in cancer immunotherapy is to rapidly generate tumor-targeted T cells, bypassing the barriers and incremental kinetics of active immunization, and eliminating MHC restriction in antigen-recognition. Once expressed in T cells, the CAR-modified T cells acquire supra-physiological properties and act as “living drugs” that may exert both immediate and long-term effects. Multiple iterations of CARs have been developed, mainly focusing on antigen-binding moiety and intracellular signaling modules, which are deemed crucial for CAR design. To achieve appropriate costimulatory signals in order to activate effector T cells, improve response, and prolong persistence, many different types of costimulatory receptors can be incorporated, alone, in tandem, or in larger arrays. However, the effect of non-signaling extracellular modules, such as hinge and transmembrane (TM) domains, on the proliferation of the transduced T cells and therapeutic efficacy of CARs remains largely unclear.

It has been reported that CAR potency is often limited, particularly in solid tumors. This is often due to low target antigen density and immune suppressive factors in the microenvironment. Consequently, there remains a need for more potent CARs to overcome these obstacles to extend the reach of these therapeutics to more diseases and to treat more patients. The invention described herein provides solutions to address these obstacles and provides additional benefits as well.

### SUMMARY

The present disclosure relates generally to the development of immuno-therapeutics, including enhanced polypeptides and chimeric antigen receptors (CARs), as well as pharmaceutical compositions comprising the same for use in treating various conditions, such as diseases (e.g., cancer). As described in greater detail below, various modifications of the hinge domain (a.k.a. hinge region) have been found to have dramatic effects on the CAR's potency and recognition of low antigen density. In particular, it has been determined that incorporation of a CD28 hinge domain in a polypeptide or CAR that either contains no costimulatory domain or contains a costimulatory domain not derived from CD28 could result in surprisingly enhanced functionality. Furthermore, experimental results described herein have demonstrated that CARs with a CD28 hinge domain outperform other products on the market.

In one aspect, provided herein are various chimeric polypeptides including: (i) a first polypeptide segment including an extracellular domain (ECD) capable of binding an antigen; (ii) a second polypeptide segment including a hinge domain derived from CD28; (iii) a third polypeptide segment including a transmembrane domain (TMD); and (iv) optionally a fourth polypeptide segment including an intracellular signaling domain (ICD) including one or more costimulatory domains, wherein the one or more costimulatory domains is not from CD28.

Non-limiting exemplary embodiments of the disclosed chimeric polypeptide of the disclosure include one or more of the following features. In some embodiments, the ICD further comprises a CD3ζ ICD. In some embodiments, the chimeric polypeptide is a chimeric antigen receptor (CAR). In some embodiments, the antigen is a tumor-associated antigen or a tumor-specific antigen. In some embodiments, the antigen is selected from the group consisting of Glypican 2 (GPC2), human epidermal growth factor receptor 2 (Her2/neu), CD276 (B7-H3), IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD19, CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, IgG Kappa light chain, IgA Lamba light chain, CD16/FcγRIII, CD64, FITC, CD22, CD27, CD30, CD70, GD2 (ganglioside G2), GD3, EGFRvIII (epidermal growth factor variant III), epidermal growth factor receptor (EGFR) and isovariants thereof, TEM-8, sperm protein 17 (Sp17), mesothelin, PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is expressed at low density.

In some embodiments, the antigen is GPC2, Her2/neu, CD276 (B7-H3), or IL-13-receptor alpha. In some embodiments, the costimulatory domain is selected from the group consisting of a costimulatory 4-1BB (CD137) polypeptide sequence, a costimulatory CD27 polypeptide sequence, a costimulatory OX40 (CD134) polypeptide sequence, a costimulatory inducible T-cell costimulatory (ICOS) polypeptide sequence, and a CD2 costimulatory domain. In some embodiments, the costimulatory domains includes a costimulatory 4-1BB (CD137) polypeptide sequence. In some embodiments, the TMD is derived from a CD28 TMD, a CD8a TMD, a CD3 TMD, a CD4 TMD, a CTLA4 TMD, and a PD-1 TMD.

In some embodiments, the chimeric polypeptide includes, in N-terminal to C-terminal direction: (i) an ECD capable of binding CD19 antigen; (ii) a hinge domain derived from CD28; (iii) a TMD derived from CD28, CD8, CD3, CD4, CTLA4, or PD-1; (iv) an ICD including a costimulatory domain from 4-1BB; and (v) a CD3ζ domain. In some embodiments, the chimeric polypeptide includes, in N-terminal to C-terminal direction: (i) an ECD capable of binding CD19 antigen; (ii) a hinge domain derived from CD28; (iii) a TMD is derived from CD8; (iv) an ICD including a costimulatory domain from 4-1BB; and (v) a CD3ζ domain. In some embodiments, the chimeric polypeptide includes, in N-terminal to C-terminal direction: (i) an ECD capable of binding CD19 antigen; (ii) a hinge domain derived from CD28; (iii) a TMD from CD8; and (iv) a CD3ζ domain.

In some embodiments, the chimeric polypeptide includes, in N-terminal to C-terminal direction: (i) an ECD capable of binding HER2 antigen; (ii) a hinge domain derived from CD28; (iii) a TMD from CD28, CD8, CD3, CD4, CTLA4, or PD-1; (iv) an ICD including a costimulatory domain from 4-1BB; and (v) a CD3ζ domain.

In some embodiments, the chimeric polypeptide includes, in N-terminal to C-terminal direction: (i) an ECD capable of binding GPC2 antigen; (ii) a hinge domain from CD28; (iii) a TMD from CD28, CD8, CD3, CD4, CTLA4, or PD-1; (iv) an ICD including a costimulatory domain from 4-1BB; and (v) a CD3ζ domain. In some embodiments, the chimeric polypeptide includes, in N-terminal to C-terminal direction: (i) an ECD capable of binding B7-H3 antigen; (ii) a hinge domain from CD28; (iii) a TMD from CD8, CTLA4, or PD-1; (iv) an ICD including a costimulatory domain from 4-1BB; and (v) a CD3ζ domain.

In some embodiments, the chimeric polypeptide has an amino acid sequence having at least 80% sequence identity to an amino acid sequence selected from the group consisting of SEQ ID NO: 13, SEQ ID NO: 27, SEQ ID NO: 39, SEQ ID NO: 53, and SEQ ID NO: 67.

In another aspect, provided herein are various recombinant nucleic acid molecules including nucleic acid sequences encoding the chimeric polypeptide as disclosed herein. Non-limiting exemplary embodiments of the recombinant nucleic acid molecules include one or more of the following features. In some embodiments, the nucleic acid sequence encodes a chimeric polypeptide. In some embodiments, the chimeric polypeptide is a CAR. In some embodiments, the recombinant nucleic acid molecule includes a nucleic acid sequence encoding a chimeric polypeptide that includes (i) an ECD capable of binding an antigen; (ii) a hinge domain derived from CD28; (iii) a TMD; and (iv) an ICD including one or more costimulatory domains, wherein the one or more costimulatory domains is not from CD28. In some embodiments, the nucleic acid sequence further encodes a CD3ζ domain. In some embodiments, the antigen is a tumor associated-antigen or a tumor-specific antigen. In some embodiments, the antigen is Glypican 2 (GPC2), human epidermal growth factor receptor 2 (Her2/neu), CD276 (B7-H3), or IL-13-receptor alpha. In some embodiments, the costimulatory domain is selected from the group consisting of a costimulatory 4-1BB (CD137) polypeptide sequence, a costimulatory CD27 polypeptide sequence, a costimulatory OX40 (CD134) polypeptide sequence, a costimulatory inducible T-cell costimulatory (ICOS) polypeptide sequence, and a CD2 costimulatory domain. In some embodiments, the costimulatory domains includes a costimulatory 4-1BB (CD137) polypeptide sequence. In some embodiments, the TMD is derived from a CD28 TMD, a CD8a TMD, a CD3 TMD, a CD4 TMD, a CTLA4 TMD, and a PD-1 TMD.

In some embodiments, the recombinant nucleic acid molecule includes a nucleic acid sequence encoding a chimeric polypeptide that includes, in N-terminal to C-terminal direction: (i) an ECD capable of binding CD19 antigen; (ii) a hinge domain derived from CD28; (iii) a TMD derived from CD8, CD28, CD3, CD4, CTLA4, or PD-1; (iv) an ICD including a costimulatory domain from 4-1BB; and (v) a CD3ζ domain. In some embodiments, the recombinant nucleic acid molecule includes a nucleic acid sequence encoding a chimeric polypeptide that includes, in N-terminal to C-terminal direction: (i) an ECD capable of binding CD19 antigen; (ii) a hinge domain derived from CD28; (iii) a TMD is derived from CD8; (iv) an ICD including a costimulatory domain from 4-1BB; and (v) a CD3ζ domain. In some embodiments, the recombinant nucleic acid molecule includes a nucleic acid sequence encoding a chimeric polypeptide that includes, in N-terminal to C-terminal direction: (i) an ECD capable of binding CD19 antigen; (ii) a hinge domain derived from CD28; (iii) a TMD from CD8; and (iv) a CD3ζ domain.

In some embodiments, the recombinant nucleic acid molecule includes a nucleic acid sequence encoding a chimeric polypeptide that includes, in N-terminal to C-terminal direction: (i) an ECD capable of binding HER2 antigen; (ii) a hinge domain derived from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant nucleic acid molecule includes a nucleic acid sequence encoding a chimeric polypeptide that includes, in N-terminal to C-terminal direction: (i) an ECD capable of binding GPC2 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain. In some embodiments, the recombinant nucleic acid molecule includes a nucleic acid sequence encoding a chimeric polypeptide that includes, in N-terminal to C-terminal direction: (i) an ECD capable of binding B7-H3 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant nucleic acid molecule includes a nucleic acid sequence encoding a chimeric polypeptide that has an amino acid sequence having at least 80% sequence identity to an amino acid sequence selected from the group consisting of SEQ ID NO: 13, SEQ ID NO: 27, SEQ ID NO: 39, SEQ ID NO: 53, and SEQ ID NO: 67. In some embodiments, the nucleic acid sequence has at least 80% sequence identity to a nucleic acid sequence selected from the group consisting of SEQ ID NO: 14, SEQ ID NO: 28, SEQ ID NO: 40, SEQ ID NO: 54, and SEQ ID NO: 68. In some embodiments, the recombinant nucleic acid molecule is operably linked to a heterologous nucleic acid sequence. In some embodiments, the recombinant nucleic acid molecule is further defined as an expression cassette in a vector. In some embodiments, the vector is a plasmid vector. In some embodiments, the vector is a viral vector. In some embodiments, the viral vector is derived from a lentivirus, an adeno virus, an adeno-associated virus, a baculovirus, or a retrovirus.

In another aspect, some embodiments of the disclosure relate to a recombinant cell including: (a) a chimeric polypeptide as described herein; and/or a nucleic acid molecule according as described herein. In some embodiments, the recombinant cell is a eukaryotic cell. In some embodiments, the recombinant cell is an immune system cell. In some embodiments, the immune system cell is a T lymphocyte.

In another aspect, some embodiments disclosed herein relate to methods for making a recombinant cell, wherein the method includes (a) providing a host cell capable of protein expression; and (b) transducing the provided host cell with a recombinant nucleic acid of the disclosure to produce a recombinant cell. Accordingly, in a related aspect, also provided herein are recombinant cells produced by the methods of the disclosure. In a further related aspect, some embodiments of the disclosure provide cell cultures that include at least one recombinant cell of the disclosure and a culture medium.

In another aspect, some embodiments of the disclosure relate to a pharmaceutical composition including a pharmaceutically acceptable carrier and one or more of: (a) a chimeric polypeptide of the disclosure; (b) a nucleic acid molecule of the disclosure; and/or (c) a recombinant cell of the disclosure. In some embodiments, the composition includes a recombinant nucleic acid of the disclosure and a pharmaceutically acceptable carrier. In some embodiments, the recombinant nucleic acid is encapsulated in a viral capsid or a lipid nanoparticle. In some embodiments, the composition includes a recombinant cell of the disclosure and a pharmaceutically acceptable carrier.

In yet another aspect, some embodiments of the disclosure relate to methods for preventing and/or treating a condition in a subject in need thereof, wherein the methods include administering to the subject a composition including one or more of the following: (a) a chimeric polypeptide of the disclosure, (b) a recombinant nucleic acid of the disclosure, (c) a recombinant cell of the disclosure, and (d) a pharmaceutical composition of the disclosure. Exemplary embodiments of the disclosed methods include one or more of the following features. In some embodiments, the condition is a proliferative disease. In some embodiments, the proliferative disease is a cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the administered composition confers increased production of interferon gamma (IFNγ) and/or interleukin-2 (IL-2) in the subject. In some embodiments, the administered composition inhibits tumor growth or metastasis of the cancer in the subject.

In some embodiments, the composition is administered to the subject individually as a first therapy or in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

In another aspect, some embodiments of the disclosure provide various kits for the practice of the methods disclosed herein. Some embodiments relate to kits for methods of the diagnosis, prevention, and/or treatment of a condition in a subject in need thereof, wherein the kits include one or more of: a chimeric polypeptide of the disclosure; a recombinant nucleic acid of the disclosure; a recombinant cell of the disclosure, and a pharmaceutical composition of the disclosure.

In another aspect, provided herein is the use of one or more of: a chimeric polypeptide of the disclosure, a recombinant nucleic acid of the disclosure, a recombinant cell of the disclosure, and a pharmaceutical composition, for the diagnosis, prevention, and/or treatment of a condition. In some embodiments, the condition is a proliferative disease. In some embodiments, the proliferative disease is a cancer.

In another aspect, provided herein is the use of one or more of the following: a chimeric polypeptide of the disclosure, a recombinant nucleic acid of the disclosure, a recombinant cell of the disclosure, or a pharmaceutical composition of the disclosure, in the manufacture of a medicament for the prevention and/or treatment of a health condition. In some embodiments, the condition is a proliferative disease. In some embodiments, the proliferative disease is a cancer.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 shows schematic diagrams of currently FDA approved clinical anti-CD19 chimeric antigen receptors.

FIGS. 2A-2B graphically summarize the results of experiments demonstrating that integration of the CD28 hinge into a CD19 CAR (CD19-28Hi-28TM-41BBz) resulted in enhancement of killing CD19^{low} cells and cytokine production in response to a range of CD19 antigen densities compared to CD19-CD8Hi-CD8TM-41BBz (Kymriah), comparing favorably to a CD19-28z CAR (Axi-Cel). FIG. 2A: NALM6 clones expressing 963 molecules of surface CD19 were co-cultured at a 1:1 ratio with either CD19-CD28ζ, CD19-4-1BBζ, or CD19-CD28H/T-4-1BBζ CAR T cells and tumor cell killing was measured in an Incucyte assay. Representative of three experiments with different T cell donors. Statistical analysis performed with repeated measures ANOVA. FIG. 2B: CD19-CD28ζ, CD19-4-1BBζ, or CD19-CD28H/T-4-1BBζ CAR T cells were co-cultured with NALM6 clones expressing various amounts of CD19 for 24 hours and IL-2 was measured in the supernatant by ELISA. Representative of three experiments with different T cell donors. Statistical comparisons performed by the student's t-test (two sided) between CD19-4-1BBζ and CD19-CD28H/T-4-1BBζ CAR T cells.

FIGS. 3A-3B schematically summarize the results of experiments suggesting that CD19-CD28Hi-CD28TM-41BBz possessed better functionality compared to CD19-CD8Hi-CD8TM-41BBz for low antigen density as determined using in vivo model of CD19^{low} leukemia. FIG. 3A: One million NALM6-CD^{192,053} cells were engrafted into NSG mice by tail vein injection. Four days later, mice were injected with 3 million CD19-CD28ζ, CD19-4-1BBζ, or CD19-CD28H/T-4-1BBζ CAR T cells. Tumor progression was measured by bioluminescence photometry and flux values (photons per second) were calculated using Living Image software. Quantified tumor flux values for individual mice are shown. Statistical analysis performed with repeated measures ANOVA. FIG. 3B: Mouse survival curves for mice as treated in FIG. 3A. Statistical analysis performed with the log-rank test. The results presented in FIGS. 3A-3B are representative of three experiments with different T cell donors (n=5 mice per group).

FIGS. 4A-4B graphically summarize the results of experiments suggesting that CD19-CD28Hi-CD28TM-41BBz possessed better functionality compared to CD19-CD8Hi-CD8TM-41BBz in normal (native) antigen density, as determined by an in vivo stress test model in which leukemia bearing mice are treated with a sub-therapeutic dose of CAR T cells. FIG. 4A: One million NALM6-wild-type cells were engrafted into NSG mice by tail vein injection. Three days later, mice were injected with 2.5×10⁵ CD19-CD28ζ, CD19-4-1BBζ, or CD19-CD28H/T-4-1BBζ CAR T cells. Tumor progression was measured by bioluminescence photometry and flux values (photons per second) were calculated using Living Image software. Quantified tumor flux values for individual mice are shown. Statistical analysis performed with repeated measures ANOVA. FIG. 4B: Mouse survival curves for mice as treated in (f). Statistical analysis performed with the log-rank test. The results presented in FIGS. 4A-4B are representative of two experiments with different T cell donors (n=5 mice per group).

FIGS. 5A-5E schematically summarize the results of experiments performed to assess functionality of CARs targeting CD19 in spleen and bone marrow tissues. One million NALM6-wild-type cells were engrafted into NSG mice by tail vein injection. Three days later, mice were injected with 5 million CD19-CD28ζ, CD19-4-1BBζ, or CD19-CD28H/T-4-1BBζ CAR T cells. The spleens (FIGS. 5A-5C) and bone marrow (FIGS. 5D-5E) of treated mice (n=5 per group) were obtained at Day +9, +16, and +29 (spleens only shown for day +29) post CAR T cell treatment. Presence of CAR positive T cells was assessed by flow cytometry. Performed one time (n=5 per CAR construct per timepoint). Statistical comparisons performed by Mann Whitney between the indicated groups. For in vitro experiments, error bars represent SD and for in vivo experiments, error bars represent SEM. p<0.05 was considered statistically significant, and p values are denoted with asterisks as follows: p>0.05, not significant, NS; * p<0.05, ** p<0.01, *** p<0.001, and **** p<0.0001.

FIGS. 6A-6C schematically summarize the results of experiments performed to assess functionality of CARs targeting Her2 in a variety of tumor models and CAR architectures in vivo. FIG. 6A is a schematic of a Her2 CAR containing a CD28 hinge-transmembrane region and 4-1BB costimulatory domain (Her2-CD28H/T-4-1BBζ). FIG. 6B: One million 143b osteosarcoma cells were orthotopically implanted in the hind leg of NSG mice. After seven days, mice were treated with 10 million Her2-4-1BBζ CAR T cells, Her2-CD28H/T-4-1BBζ CAR T cells, or untransduced control T cells (MOCK). Leg measurements were obtained twice weekly with digital calibers. Measurements for individual mice are shown. Statistical analysis performed with repeated measures ANOVA. FIG. 6C: Survival curves for mice treated as in FIG. 6B: Statistical analysis performed with the log-rank test. The results presented in FIGS. 6B-6C are representative of two experiments with different T cell donors (n=5 mice per group).

FIGS. 7A-7D schematically summarize the results of experiments performed to assess functionality of CARs targeting B7-H3 in a variety of tumor models and CAR architectures. FIG. 7A depicts a schematic of a B7-H3 CAR containing a CD28 hinge-transmembrane region and 4-1BB costimulatory domain (B7-H3-CD28H/T-4-1BBζ). FIG. 7B: One million CHLA255 neuroblastoma cells were engrafted into NSG mice by tail vein injection in a metastatic neuroblastoma model. Six days later, mice were injected with 10 million B7-H3-4-1BBζ CAR T cells, B7-H3-CD28H/T-4-1BBζ CAR T cells, or untransduced control T cells (MOCK). Tumor progression was measured by bioluminescence photometry and flux values (photons per second) were calculated using Living Image software. Representative bioluminescent images are shown. FIG. 7C: Quantified tumor flux values for individual mice treated as in FIG. 7B. Statistical analysis performed with repeated measures ANOVA. FIG. 7D: Survival curves for mice treated as in FIG. 7B. Statistical analysis performed with the log-rank test. The results presented in FIGS. 7B-7D are representative of two experiments with different T cell donors. For in vitro experiments, error bars represent SD and for in vivo experiments, error bars represent SEM. p<0.05 was considered statistically significant, and p values are denoted with asterisks as follows: p>0.05, not significant, NS; * p<0.05, ** p<0.01, *** p<0.001, and **** p<0.0001.

FIGS. 8A-8C graphically summarizes the results of experiments suggesting that the CD28 hinge domain is responsible for enhancement in CAR T cell efficacy even in the absence of costimulation (in a first generation CAR construct). FIG. 8A: is a schematic of exemplary first generation CD19 CARs with either a CD8 or CD28 hinge-transmembrane region (CD19-CD8H/T-ζ and CD19-CD28H/T-ζ). FIG. 8B: NALM6 clones expressing either 963 or 45,851 molecules of surface CD19 were co-cultured at a 1:1 ratio with either CD19-CD28ζ, CD19-4-1BBζ, CD19-CD28H/T-ζ or CD19-CD8H/T-ζ CAR T cells and tumor cell killing was measured in an Incucyte assay. Representative of three experiments with different T cell donors. Statistical analysis performed with repeated measures ANOVA between CD19-CD28H/T-ζ and CD19-CD8H/T-ζ. FIG. 8C: CD19-CD28ζ, CD19-4-1BBζ, CD19-CD28H/T-ζ, and CD19-CD8H/T-4 CAR T cells were co-cultured with NALM6 clones expressing various amounts of CD19 for 24 hours and secreted IL-2 was measured in the supernatant by ELISA. Representative of three experiments with different T cell donors. Statistical comparisons performed with the student's t-test (two sided) between CD19-CD28H/T-ζ and CD19-CD8H/T-ζ.

FIGS. 9A-9D depict schematic structures of four exemplary CAR designs in accordance with some embodiments of the disclosure.

FIGS. 10A-10B are flow plots showing the expression of the CAR designs described in FIGS. 9A-9D. All CARs expressed similarly on the surface of T cells, regardless of the hinge and transmembrane domains.

FIGS. 11A-11B schematically summarize the results of experiments suggesting that the CD28 hinge domain is responsible for the enhancement in CAR functionality, and further suggesting that the CD28Hi-CD8TM combination can be a more potent version. FIG. 11A: IFNγ production in response to co-culture with NALM6 clones expressing increasing amounts of CD19. FIG. 11B: production of cytokine IL-2 in response to co-culture with NALM6 clones expressing increasing amounts of CD19.

FIG. 12 schematically summarizes the results of experiments suggesting that the CD28 hinge domain is responsible for the enhancement in cell-killing efficacy against CD19^{low} leukemia.

FIGS. 13A-13C pictorially summarize the results of experiments performed to illustrate that the CD28 Hinge-TMD results in more efficient receptor clustering, T cell activation, and tumor cell killing. FIGS. 13A-13B: CAR T cells and NALM6 cells were seeded at low density on a microwell plate and scanned for wells containing one tumor cell and one CAR T cell. Experiment was performed 6 times across two different T cell donors. FIG. 13A: A representative well from the single-cell microwell killing experiment is shown. CAR T cells and NALM6 leukemia cells were distinguished by CellTrace Far Red (false-colored magenta) and GFP (false-colored cyan) labels, respectively. Cell death was determined by influx of cell-impermeable propidium iodide dye (PI, false-colored yellow). Lytic conjugates were defined as events where one T cell and one NALM6 cell remained within a threshold distance, and the NALM6 cell died (took up PI). Nonlytic conjugates represent conjugates where the T cell and tumor cell interact but the NALM6 cell did not die (did not take up PI). DIC: Differential interference contrast and Epi: epifluorescence. FIG. 13B: Time from T cell/tumor cell interaction to PI influx was measured in wells containing one tumor cell and one T cell per CAR construct. Pooled data from all 6 experiments (400-600 wells) is shown. Error bars represent SD. Statistical analysis performed with the student's t-test (two sided). FIG. 13C: The fraction of nonlytic conjugates (conjugates where the T cell and tumor cell interacted but the NALM6 cell did not die) that resulted in T cell death was measured in each of six experiments.

FIGS. 14A-14I schematically summarize the results of additional experiments performed to illustrate that the CD28 Hinge-TMD results in more efficient receptor clustering, T cell activation, and tumor cell killing especially when target antigen density is low. FIG. 14A: Diagram of TIRF (Total Internal Reflection Fluorescence) imaging. To stimulate CD19-CD28H/T-4-1BBζ and CD19-4-1BBζ CART cells, CAR T cells were exposed to a planar supported lipid bilayer (SLB) functionalized with a freely diffusing CD19 proteins coupled by a biotin-streptavidin-biotin bridge. Ligand-receptor engagement leads to the reorganization of ligand-bound receptors into microclusters that recruit the tyrosine kinase ZAP70 (fused to GFP, not shown in this diagram) from the cytosol to the plasma membrane, and drive the centripetal translocation of the microclusters from the periphery to the cell center. These events are visualized by TIRF microscopy (fluorescence: CAR-mCherry, ZAP70-GFP, Streptavidin-Alexa647). Ligand density in the planar supported lipid bilayer is controlled through the concentration of Biotin-PE containing small unilamellar vesicles (SUVs). To assess the level of recruitment/degree of clustering across cells that display a range of expression levels, index of dispersion (i.e., normalized variance, which equals the standard deviation divided by the mean of the fluorescence intensity of each cell, see methods for details) was used. FIG. 14B: Degree of clustering (index of dispersion) for CAR molecules recruited to the immune synapse for each CAR construct at different CD19 densities in the experiment in FIGS. 14A-14I. FIG. 14C: Representative images of single CD19-CD28H/T-4-1BBζ-mCherry (left panels) and CD19-CD8H/T-4-1BBζ-mCherry (right panels) CAR T cells transduced with ZAP70-GFP activated on planar supported lipid bilayer containing high (˜6.0 molecule/μm²; top panel) and low (˜0.6 molecule/μm2; bottom panel) concentrations of CD19. FIG. 14D: Degree of clustering (index of dispersion) for ZAP70-GFP recruited to the immune synapse for each CAR construct at four different CD19 densities. FIG. 14E: Pooled ZAP70 degree of clustering (index of dispersion) data from FIG. 14D plotted as a dose response curve for ligand density. FIG. 14F: Percentage of cells activated (ZAP70 recruitment above a threshold) plotted as a dose response curve for ligand density. FIG. 14G: Degree of clustering (index of dispersion) for ligand-receptor complexes recruited to the immune synapse for each CAR construct at four different CD19 densities. FIG. 14H: Pooled ligand-receptor complex degree of clustering (index of dispersion) data from (h) plotted as a dose response curve for ligand density. FIG. 14I: Percentage of cells recruiting ligand-receptor complexes (above a threshold) plotted as a dose response curve for ligand density. The results presented in FIGS. 14A-14I (shown as mean±SD) are representative from one experiment of two performed with different T cell donors. n>100 per condition. Statistical analysis performed with the two-tailed t-test. p<0.05 was considered statistically significant, and p values are denoted with asterisks as follows: p>0.05, not significant, NS; * p<0.05, ** p<0.01, *** p<0.001, and **** p<0.0001. Data are representative from two experiments with different T cell donors. n>100 per condition. Statistical analysis performed with the student's t-test.

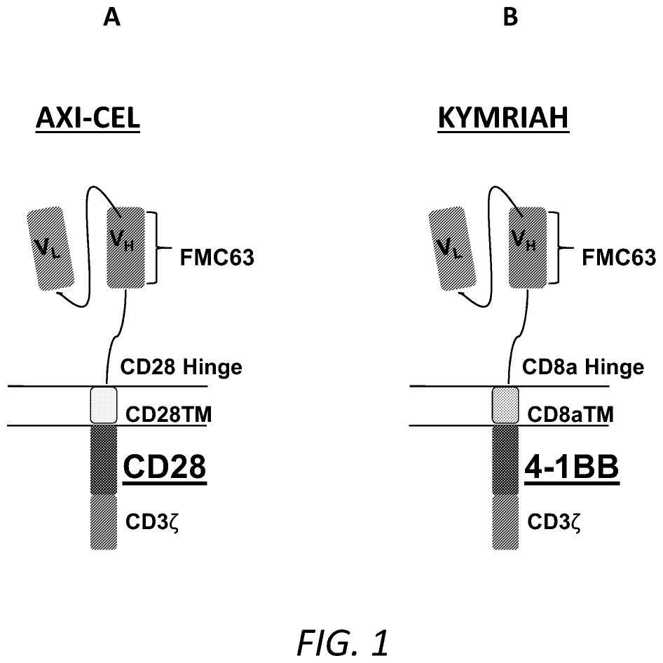

### DETAILED DESCRIPTION OF THE DISCLOSURE

The present disclosure relates generally to, inter alia, chimeric polypeptides and chimeric antigen receptors (CARs) that include a hinge domain from CD28 and optionally a costimulatory domain heterologous with respect to the CD28 hinge domain, e.g., a costimulatory domain that is not from CD28. Various chimeric polypeptides and CARs disclosed herein do not contain a costimulatory domain, whereas other versions of the chimeric polypeptides and CARs disclosed herein contain one or more costimulatory domains which are not from CD28. The disclosure also provides compositions and methods useful for making such polypeptides and CARs, as well as methods for the detection and treatment of conditions, such as diseases (e.g., cancer).

Chimeric antigen receptors are recombinant receptor constructs which, in their usual format, graft the specificity of an antibody to the effector function of a T cell. Within a chimeric antigen receptor, the hinge domain generally refers to a polypeptide structure positioned between the targeting moiety and the T cell plasma membrane, i.e., disposed between the targeting moiety and the intracellular domain. These sequences are generally derived from IgG subclasses (such as IgG1 and IgG4), IgD and CD8 domains, of which IgG1 has been most extensively used. In recent years, several studies of the hinge domain mainly focused on the following aspects: (1) reducing binding affinity to the Fcγ receptor, thereby eliminating certain types of off-target activation; (2) enhancing the single-chain variable fragment (scFv) flexibility, thereby relieving the spatial constraints between particular epitopes targeted on tumor antigens and the CAR's antigen-targeting moiety; (3) reducing the distance between an scFv and the target epitope(s); and (4) facilitating the detection of CAR expression using anti-Fc reagents. Nevertheless, the influences of the hinge domain on CAR T cell physiology are not well understood.

As described in greater detail below, to better understand the effect of a hinge domain on CAR T cells, several versions of CARs, without or with a hinge domain derived from CD8a or CD28 have been designed and constructs. Subsequently, the effect of the presence or absence of the hinge domains on the growth kinetics, cytokine production, and cytotoxicity of CAR T cells ex vivo and in vivo has been systematically evaluated. It has been then determined that the incorporation of a CD28 hinge domain into CAR constructs can substantially enhance cell killing, enhance production of cytokines, e.g., IFNγ and interleukin-2 (IL-2) in response to tumor. In addition, it was also found that anti-CD19 CAR T cells with or without a CD28 hinge domain have similar expression levels, whereas a CD28 hinge domain can enhance the in vivo antitumor activity of anti-CD19 CART cells.

The experimental results presented herein demonstrate that a CD28 hinge domain incorporated in several CAR designs was capable of increasing the antitumor efficacy of the corresponding CAR T cells. These results suggest potential novel strategies in designing more effective chimeric antigen receptors to complement existing immunotherapeutic approaches.

Nucleic acid molecules encoding these polypeptides and CARs are also provided. The disclosure also provides compositions and methods useful for producing such polypeptides and CARs, as well as methods for the prevention and/or treatment of conditions, such as cancer.

All publications and patent applications mentioned in this disclosure are herein incorporated by reference to the same extent as if each individual publication or patent application was specifically and individually indicated to be incorporated by reference.

### General Experimental Procedures

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference. As appropriate, procedures involving the use of commercially available kits and reagents are generally carried out in accordance with manufacturer defined protocols and/or parameters unless otherwise noted.

### Definition

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

As used herein, the term “antibody” refers to a class of proteins that are generally known as immunoglobulins that specifically bind to an antigen molecule. The term antibody includes full-length monoclonal antibodies (mAb), such as IgG2 monoclonal antibodies, which include immunoglobulin Fc regions. The term antibody also includes bispecific antibodies, diabodies, single-chain antibody fragments (scFv), and antibody fragments such as Fab, F(ab′)2, and Fv. In instances where the antibody is a bispecific antibody, the bispecific antibody can be in many different formats. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. As such, antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target (e.g., CD19, GPC2, or HER2) is maintained.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, the term “chimeric antigen receptor” (CAR) refers to a polypeptide construct comprising at least an extracellular antigen-binding domain, a TMD and a cytoplasmic signaling domain (also referred to as “an intracellular signaling domain” or ICD). In some cases, the cytoplasmic signaling domain includes a functional signaling domain derived from a stimulatory molecule. The stimulatory molecule often is the zeta chain associated with the T cell receptor complex. Optionally, the ICD can further include one or more functional signaling domains derived from at least one costimulatory molecule, such as e.g., 4-1BB (i.e., CD137), CD27, and/or CD28.

Generally, the CARs of the disclosure include an ectodomain and an endodomain each as defined by the host cell wall. In this regard, the terms “ectodomain” or “extracellular domain” generally refer to the portion of the CAR polypeptide outside of the cell or exterior to the membranous lipid bilayer, which may include the antigen recognition binding domains, an optional hinge domain, and any spacer domains exterior to the amino acid residues physically spanning the membrane. Conversely, the terms “endodomain” or “intracellular domain” generally refer to the portion of the CAR polypeptide inside the cell or interior to the membranous lipid bilayer, which may also include any spacer domains interior to the amino acid residues physically spanning the membrane, as well as the ICD, which comprises one or more costimulatory signaling domains (e.g., ITAM-containing sequences, costimulatory domains, etc.).

One skilled in the art will understand that the term “derived from” when used in reference to a nucleic acid or polypeptide molecule refers to the origin or source of the molecule, and may include naturally occurring, recombinant, unpurified, or purified molecules. Nucleic acid or polypeptide molecules are considered “derived from” when they include portions or elements assembled in such a way that they produce a functional unit. The portions or elements can be assembled from multiple sources provided that they retain evolutionarily conserved function. In some embodiments, the derivative nucleic acid or polypeptide molecules include substantially the same sequence as the source nucleic acid or polypeptide molecule. For example, the derivative nucleic acid or polypeptide molecules of the present disclosure may have at least 80%, 85%, 90%, 95%, 98%, 99%, or 100% sequence identity to the source nucleic acid or polypeptide molecule.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

The term “operably linked”, as used herein, denotes a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is a functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous. In the context of a polypeptide, “operably linked” refers to a physical linkage (e.g., directly or indirectly linked) between amino acid sequences (e.g., different domains) to provide for a described activity of the polypeptide. In the present disclosure, various domains of the recombinant polypeptides of the disclosure may be operably linked to retain proper folding, processing, targeting, expression, binding, and other functional properties of the recombinant polypeptides in the cell. Operably linked domains of the recombinant polypeptides of the disclosure may be contiguous or non-contiguous (e.g., linked to one another through a linker).

The term “percent identity” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. The amino acid substitution(s) may be a conservative amino acid substitution, for example at a non-essential amino acid residue in the CDR sequence(s). A “conservative amino acid substitution” is understood to be one in which the original amino acid residue is substituted with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “recombinant” nucleic acid molecule, polypeptide, and cell as used herein, refers to a nucleic acid molecule, polypeptide, and cell that has been altered through human intervention. As non-limiting examples, a recombinant nucleic acid molecule can be one which: 1) has been synthesized or modified in vitro, for example, using chemical or enzymatic techniques, or recombination of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. A non-limiting example of a recombinant protein is a chimeric antigen receptor as provided herein.

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

The term “vector” is used herein to refer to a nucleic acid molecule or sequence capable of transferring or transporting another nucleic acid molecule. For example, a vector can be used as a gene delivery vehicle to transfer a gene into a cell. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

As described in greater detail below, one aspect of the present disclosure relates to novel chimeric polypeptides and chimeric antigen receptors (CARs) that include a hinge domain from CD28. In some embodiments, the CARs of the disclosure further include a costimulatory domain heterologous to the CD28 hinge domain, e.g., a costimulatory domain that is not from CD28. Also provided are recombinant nucleic acids encoding such chimeric polypeptides, as well as recombinant cells that have been engineered to express a chimeric polypeptide as disclosed herein and are directed against a cell of interest such as a cancer cell.

### Chimeric Polypeptides

In one aspect, some embodiments disclosed herein relate to chimeric polypeptides which include (i) a first polypeptide segment including an ECD capable of binding an antigen; (ii) a second polypeptide segment including a hinge domain from CD28; (iii) a third polypeptide segment including a TMD. In some embodiments, the polypeptides further include a fourth polypeptide segment including an ICD including one or more costimulatory domains, wherein the one or more costimulatory domains are not from CD28. The binding of the ECD to its respective target can be either in a competitive or non-competitive fashion with a natural ligand of the target antigen. Accordingly, in some embodiments of the disclosure, the binding of the ECD to its target antigen can be ligand-blocking. In some other embodiments, the binding of the ECD to its target antigen does not block binding of the natural ligand. In some embodiments, the chimeric polypeptide includes at least one polypeptide segment operably linked to a second polypeptide segment to which it is not naturally linked in nature. The chimeric polypeptide segments may normally exist in separate proteins that are brought together in the chimeric polypeptide disclosed herein or they may normally exist in the same protein but are placed in a new arrangement in the chimeric polypeptide disclosed herein. A chimeric polypeptide as disclosed herein may be created, for example, by chemical synthesis, or by creating and translating a chimeric polynucleotide in which the polypeptide segments are encoded in the desired relationship.

Designation of the polypeptide segments of the disclosed polypeptide as the “first”, “second”, “third”, or “fourth” polypeptide segments is not intended to imply any particular structural arrangement of the “first”, “second”, “third”, or “fourth” polypeptide segments within the chimeric polypeptide. In addition or alternatively, the chimeric polypeptide may include more than one polypeptide segment capable of binding to a target antigen, and/or at least two polypeptide segments each capable of binding to the same target antigen or to a different target antigen.

In some embodiments, at least two of the polypeptide segments are directly linked to one another. In some embodiments, all of the polypeptide segments are directly linked to one another. In some embodiments, at least two of the polypeptide segments are directly linked to one another via at least one covalent bond. In some embodiments, at least two of the polypeptide segments are directly linked to one another via at least one peptide bond. In some embodiments, the chimeric polypeptides of the disclosure include one or more linkers which join the two or more polypeptide segments together. In some embodiments, at least two of the polypeptide segments are operably linked to one another via a linker. There is no particular limitation on the linkers that can be used in the chimeric polypeptides described herein. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The linker can also be a linker peptide sequence. Accordingly, in some embodiments, at least two of the polypeptide segments are operably linked to one another via a linker peptide sequence. In principle, there are no particular limitations to the length and/or amino acid composition of the linker peptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues.

### Chimeric Antigen Receptors (CARs)

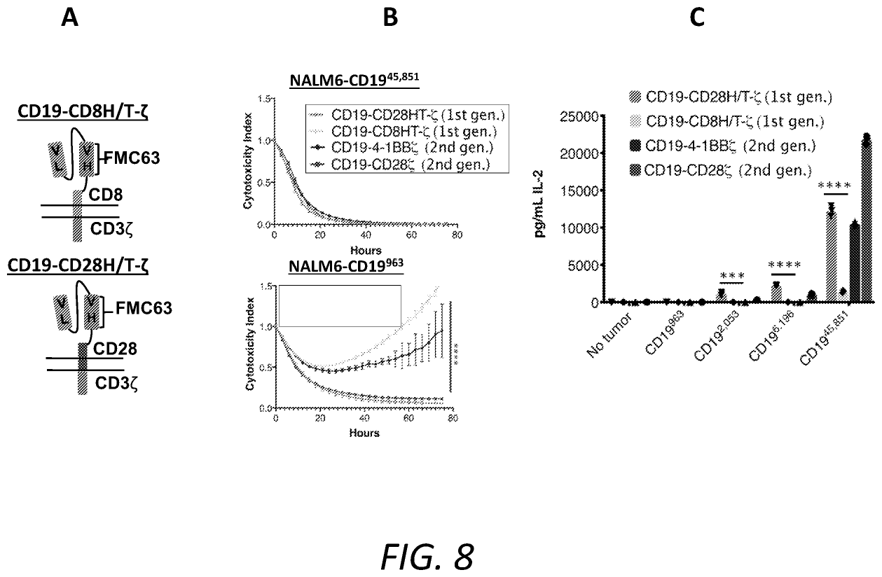

As described above, the chimeric polypeptides of the present disclosure include (i) an ECD capable of binding an antigen; (ii) a hinge domain from CD28; (iii) a TMD; and (iv) an ICD including one or more costimulatory domains, wherein the one or more costimulatory domains are not from CD28. In some embodiments, chimeric polypeptides disclosed herein are configured as chimeric antigen receptors (CARs). CARs are recombinant receptor constructs composed of an extracellular antigen-binding moiety derived from an antibody, joined to a hinge domain and a TMD, which is further linked to the intracellular T cell signaling domains of the T cell receptor. As such, CAR T cells can combine the specificity of an antibody with the cytotoxic and memory functions of T cells. In some embodiments, the disclosed CARs do not include a costimulatory domain. These CARs are referred to as first generation CARs (see, e.g., SEQ ID NO: 39 and FIG. 8A). In some embodiments, the disclosed CARs include one or more costimulatory domains, wherein the one or more costimulatory domains are not derived from CD28.

### Extracellular Domains (ECD)

In some embodiments, the ECD of the chimeric polypeptides disclosed herein has a binding affinity for one or more target ligands. In some embodiments, the target ligand is expressed on a cell surface, or is otherwise anchored, immobilized, or restrained so that it can exert a mechanical force on the chimeric polypeptides. As such, without being bound to any particular theory, binding of the ECD of a chimeric polypeptide provided herein to a cell-surface ligand does not necessarily remove the target ligand from the target cell surface, but instead enacts a mechanical pulling force on the chimeric polypeptide. For example, an otherwise soluble ligand may be targeted if it is bound to a surface, or to a molecule in the extracellular matrix. In some embodiments, the target ligand is a cell-surface ligand. Non-limiting examples of suitable ligand types include cell surface receptors, adhesion proteins, carbohydrates, lipids, glycolipids, lipoproteins, and lipopolysaccharides that are surface-bound, integrins, mucins, and lectins. In some embodiments, the ligand is a protein. In some embodiments, the ligand is a carbohydrate.

In some embodiments, the ECD of the chimeric polypeptides disclosed herein includes an antigen-binding moiety that binds to one or more target antigens. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, an antigen-binding fragment (Fab), a single-chain variable fragment (scFv), a nanobody, a diabody, a triabody, a minibody, an F(ab′)2 fragment, an F(ab) fragment, a VH domain, a VL domain, a single chain variable fragment (scFv), a single domain antibody (sdAb), a VNAR domain, and a VHH domain, or a functional fragment thereof. In some embodiments, the antigen-binding moiety includes a heavy chain variable region and a light chain variable region. In some embodiments, the antigen-binding moiety includes a scFv.

The antigen-binding moiety can include naturally-occurring amino acid sequences or can be engineered, designed, or modified so as to provide desired and/or improved properties, e.g., binding affinity. Generally, the binding affinity of an antibody or an antigen-binding moiety for a target antigen (e.g., CD19 antigen or GPC2 antigen) can be calculated by the Scatchard method described by Frankel et al., Mol. Immunol, 16: 101-106, 1979. In some embodiments, binding affinity can be measured by an antigen/antibody dissociation rate. In some embodiments, a high binding affinity can be measured by a competition radioimmunoassay. In some embodiments, binding affinity can be measured by ELISA. In some embodiments, antibody affinity can be measured by flow cytometry. An antibody that “selectively binds” a target antigen (such as CD19 or HER2) is an antibody that binds the target antigen with high affinity and does not significantly bind other unrelated antigens but binds the antigen with high affinity, e.g., with an equilibrium constant (KD) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a chimeric polypeptide of the present disclosure. For example, a chimeric polypeptide with an ECD including an antibody specific for a HER2 antigen can target cells to HER2-expressing breast cancer cells. In some embodiments, the ECD of the chimeric polypeptides disclosed herein is capable of binding a tumor-associated antigen (TAA) or a tumor-specific antigen (TSA). A skilled artisan will understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

### Antigens

In principle, there are no particular limitations with regard to suitable target antigens. In some embodiments of the disclosure, the antigen-binding moiety of the ECD is specific for an epitope present in an antigen that is expressed by a tumor cell, i.e., a tumor-associated antigen. The tumor-associated antigen can be an antigen associated with, e.g., a pancreatic cancer cell, a colon cancer cell, an ovarian cancer cell, a prostate cancer cell, a lung cancer cell, mesothelioma cell, a breast cancer cell, a urothelial cancer cell, a liver cancer cell, a head and neck cancer cell, a sarcoma cell, a cervical cancer cell, a stomach cancer cell, a gastric cancer cell, a melanoma cell, a uveal melanoma cell, a cholangiocarcinoma cell, a multiple myeloma cell, a leukemia cell, a lymphoma cell, and a glioblastoma cell. In some embodiments, the antigen-binding moiety is specific for an epitope present in a tissue-specific antigen. In some embodiments, the antigen-binding moiety is specific for an epitope present in a disease-associated antigen.

Non-limiting examples of suitable target antigens include Glypican 2 (GPC2), human epidermal growth factor receptor 2 (Her2/neu), CD276 (B7-H3), IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional antigens that can be suitable for the chimeric polypeptides and CARs disclosed herein include, but are not limited to, the dimeric form of the pyruvate kinase isoenzyme type M2 (tumor M2-PK), CD19, CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3(CD61), galactin, K-Ras (V-Ki-ras2 Kirsten rat sarcoma viral oncogene), and Ral-B. In some embodiments, the antigen is Glypican 2 (GPC2), CD19, human epidermal growth factor receptor 2 (Her2/neu), CD276 (B7-H3), or IL-13-receptor alpha.

In some embodiments, the antigen is expressed at low density on target cells, e.g., less than about 6,000 molecules of the target antigen per cell. In some embodiments, the antigen is expressed at a density of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell.

In some embodiments, the chimeric polypeptides and CARs disclosed herein include an ECD including an antigen-binding moiety that binds GPC2. In some embodiments, the chimeric polypeptides and CARs disclosed herein include an ECD including an antigen-binding moiety that binds CD19. In some embodiments, the chimeric polypeptides and CARs disclosed herein include an ECD including an antigen-binding moiety that binds HER2. In some embodiments, the chimeric polypeptides and CARs disclosed herein include an ECD including an antigen-binding moiety that binds B7-H3. In some embodiments, the chimeric polypeptides and CARs disclosed herein include an ECD including an antigen-binding moiety having an amino acid sequence exhibiting at least 80% sequence identity to SEQ ID NO: 3, SEQ ID NO: 17, SEQ ID NO: 31, SEQ ID NO: 43, or SEQ ID NO: 57. In some embodiments, the antigen-binding moiety has an amino acid sequence exhibiting at least 80%, at least 80%, at least 85%, at least 90%, at least 95%, at least 96%, at least 97%, at least 98%, at least 99%, or at least 100% sequence identity to the sequence of SEQ ID NO: 3, SEQ ID NO: 17, or SEQ ID NO: 31. In some embodiments, the antigen-binding moiety has an amino acid sequence exhibiting at least 85%, at least 90%, at least 95%, at least 96%, at least 97%, at least 98%, at least 99%, or at least 100% sequence identity to the sequence of SEQ ID NO: 43. In some embodiments, the antigen-binding moiety has an amino acid sequence exhibiting at least 85%, at least 90%, at least 95%, at least 96%, at least 97%, at least 98%, at least 99%, or at least 100% sequence identity to the sequence of SEQ ID NO: 57.

### Hinge Domains

As described above, within a chimeric antigen receptor, the term “hinge domain” generally refers to a flexible polypeptide connector region disposed between the targeting moiety and the TMD. These sequences are generally derived from IgG subclasses (such as IgG1 and IgG4), IgD and CD8 domains, of which IgG1 has been most extensively used. In some embodiments, the hinge domain provides structural flexibility to flanking polypeptide regions. The hinge domain may consist of natural or synthetic polypeptides. It will be appreciated by those skilled in the art that hinge domains may improve the function of the CAR by promoting optimal positioning of the antigen-binding moiety in relationship to the portion of the antigen recognized by the same. It will be appreciated that, in some embodiments, the hinge domain may not be required for optimal CAR activity. In some embodiments, a beneficial hinge domain comprising a short sequence of amino acids promotes CAR activity by facilitating antigen-binding by, e.g., relieving any steric constraints that may otherwise alter antibody binding kinetics. The sequence encoding the hinge domain may be positioned between the antigen recognition moiety and the TMD. In some embodiments, the hinge domain is operably linked downstream of the antigen-binding moiety and upstream of the TMD.

The hinge sequence can generally be any moiety or sequence derived or obtained from any suitable molecule. For example, in some embodiments, the hinge sequence can be derived from the human CD8a molecule or a CD28 molecule and any other receptors that provide a similar function in providing flexibility to flanking regions. The hinge domain can have a length of from about 4 amino acid (aa) to about 50 aa, e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. Suitable hinge domains can be readily selected and can be of any of a number of suitable lengths, such as from 1 amino acid (e.g., Gly) to 20 aa, from 2 aa to 15 aa, from 3 aa to 12 aa, including 4 aa to 10 aa, 5 aa to 9 aa, 6 aa to 8 aa, or 7 aa to 8 aa, and can be 1, 2, 3, 4, 5, 6, or 7 aa. Non-limiting examples of suitable hinge domains include a CD8 hinge domain, a CD28 hinge domain, a CTLA4 hinge domain, or an IgG4 hinge domain. In some embodiments, the hinge domain can include regions derived from a human CD8α (a.k.a. CD8α) molecule or a CD28 molecule and any other receptors that provide a similar function in providing flexibility to flanking regions. In some embodiments, the CAR disclosed herein includes a hinge domain derived from a CD8α hinge domain. In some embodiments, the hinge domain can include one or more copies of the CD8α hinge domain. In some embodiments, the CAR disclosed herein includes a hinge domain derived from a CD28 hinge domain. In some embodiments, the hinge domain can include one or more copies of the CD28 hinge domain. In some embodiments, the chimeric polypeptides and CARs disclosed herein include a hinge domain having an amino acid sequence exhibiting at least 80% sequence identity to the sequence of SEQ ID NO: 5, SEQ ID NO: 19, SEQ ID NO: 33, SEQ ID NO: 45, or SEQ ID NO: 59. In some embodiments, the hinge domain has an amino acid sequence exhibiting at least 85%, at least 90%, at least 95%, at least 96%, at least 97%, at least 98%, at least 99%, or at least 100% sequence identity to the sequence of SEQ ID NO: 5, SEQ ID NO: 19, SEQ ID NO: 33, SEQ ID NO: 45, or SEQ ID NO: 59.

### Costimulatory Domains

Generally, the costimulatory domain suitable for the chimeric polypeptides, e.g., CARs disclosed herein can be any one of the costimulatory domains known in the art. Examples of suitable costimulatory domains that can enhance cytokine production and include, but are not limited to, costimulatory polypeptide sequences derived from 4-1BB (CD137), CD27, CD28, OX40 (CD134), and costimulatory inducible T-cell costimulatory (ICOS) polypeptide sequences. Accordingly, in some embodiments, the costimulatory domain of the chimeric polypeptides and CARs disclosed herein is selected from the group consisting of a costimulatory 4-1BB (CD137) polypeptide sequence, a costimulatory CD27 polypeptide sequence, a costimulatory CD28 polypeptide sequence, a costimulatory OX40 (CD134) polypeptide sequence, and a costimulatory inducible T-cell costimulatory (ICOS) polypeptide sequence. In some embodiments, the chimeric polypeptides and CARs disclosed herein include a costimulatory domain derived from a costimulatory 4-1BB (CD137) polypeptide sequence. In some embodiments, the chimeric polypeptides and CARs disclosed herein include a costimulatory 4-1BB (CD137) polypeptide sequence. In some embodiments, the chimeric polypeptides and CARs disclosed herein include a costimulatory domain derived from a costimulatory CD28 polypeptide sequence. In some embodiments, the chimeric polypeptides and CARs disclosed herein include a costimulatory CD28 polypeptide sequence. In some embodiments, the chimeric polypeptides and CARs disclosed herein include a costimulatory domain having an amino acid sequence exhibiting at least 80% sequence identity to the sequence of SEQ ID NO: 9, SEQ ID NO: 23, SEQ ID NO: 49, or SEQ ID NO: 63. In some embodiments, the chimeric polypeptides and CARs disclosed herein include a costimulatory domain having an amino acid sequence exhibiting at least 85%, at least 90%, at least 95%, at least 96%, at least 97%, at least 98%, at least 99%, or at least 100% sequence identity to the sequence of SEQ ID NO: 9, SEQ ID NO: 23, SEQ ID NO: 49, or SEQ ID NO: 63.

In some embodiments of the disclosure, the ICD of the disclosed CARs includes conserved amino acid motifs that serve as substrates for phosphorylation such as, for example, immunoreceptor tyrosine-based activation motifs (ITAM), and/or immunoreceptor tyrosine-based inhibition motifs (ITIM). In some embodiments, the ICD of the disclosed CARs includes at least 1, at least 2, at least 3, at least 4, or at least 5 specific tyrosine-based motifs selected from ITAM motifs, an ITIM motifs, or related intracellular motifs that serve as a substrate for phosphorylation. In some embodiments of the disclosure, the ICD of the disclosed CARs includes at least 1, at least 2, at least 3, at least 4, or at least 5 ITAMs. Generally, any ICD including an ITAM can be suitably used for the construction of the chimeric polypeptides as described herein. An ITAM generally includes a conserved protein motif that is often present in the tail portion of signaling molecules expressed in many immune cells. The motif may include two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some embodiments, the ICD comprising at least 1, at least 2, at least 3, at least 4, or at least 5 ITAMs independently selected from the ITAMs derived from CD3ζ, FcRγ, and combinations thereof. In some embodiments, the ICDs of the disclosed CARs comprises a CD3ζ ICD. In some embodiments, the chimeric polypeptides and CARs disclosed herein include a CD3ζ ICD having an amino acid sequence exhibiting at least 80% sequence identity to the sequence of SEQ ID NO: 11, SEQ ID NO: 25, SEQ ID NO: 37, SEQ ID NO: 51, or SEQ ID NO: 65. In some embodiments, the chimeric polypeptides and CARs disclosed herein include a CD3ζ ICD having an amino acid sequence exhibiting at least 85%, at least 90%, at least 95%, at least 96%, at least 97%, at least 98%, at least 99%, or at least 100% sequence identity to the sequence of SEQ ID NO: 11, SEQ ID NO: 25, SEQ ID NO: 37, SEQ ID NO: 51, or SEQ ID NO: 65.

### Transmembrane Domains (MD)

Generally, the transmembrane domain (also referred to as transmembrane region) suitable for the chimeric polypeptides and CARs disclosed herein can be any one of the TMDs known in the art. Without being bound to theory, it is believed that the TMD traverses the cell membrane, anchors the CAR to the cell surface, and connects the ECD to the ICD, thus impacting expression of the CAR on the cell surface. Examples of suitable TMDs include, but are not limited to, a CD28 TMD, a CD8α TMD, a CD3 TMD, a CD4 TMD, a CTLA4 TMD, and a PD-1 TMD. Accordingly, in some embodiments, the TMD is derived from a CD28 TMD, a CD8α TMD, a CD3 TMD, a CD4 TMD, a CTLA4 TMD, and a PD-1 TMD. In some embodiments, the TMD includes a CD28 TMD, a CD8α TMD, a CD3 TMD, a CD4 TMD, a CTLA4 TMD, and a PD-1 TMD. In some embodiments, the chimeric polypeptides and CARs disclosed herein include a TMD derived from a CD8α. In some embodiments, the chimeric polypeptides and CARs disclosed herein include a CD8α TMD. In some embodiments, the chimeric polypeptides and CARs disclosed herein include a TMD derived from a CD28. In some embodiments, the chimeric polypeptides and CARs disclosed herein include a CD28 TMD. In some embodiments, the chimeric polypeptides and CARs disclosed herein include a TMD an amino acid sequence exhibiting at least 80% sequence identity to the sequence of SEQ ID NO: 7, SEQ ID NO: 21, SEQ ID NO: 35, SEQ ID NO: 47, or SEQ ID NO: 61. In some embodiments, the chimeric polypeptides and CARs disclosed herein include a TMD an amino acid sequence exhibiting at least 85%, at least 90%, at least 95%, at least 96%, at least 97%, at least 98%, at least 99%, or at least 100% sequence identity to the sequence of SEQ ID NO: 7, SEQ ID NO: 21, SEQ ID NO: 35, SEQ ID NO: 47, or SEQ ID NO: 61. In some embodiments, the ICD includes a CD3ζ ICD which, without being bound to any particular theory, is believed to mediate downstream signaling during T cell activation.

### Extracellular Spacer

In some embodiments, the CARs disclosed herein further include an extracellular spacer domain including one or more intervening amino acid residues that are positioned between the ECD and the hinge domain. In some embodiments, the extracellular spacer domain is operably linked downstream to the ECD and upstream to the hinge domain. In principle, there are no particular limitations to the length and/or amino acid composition of the extracellular spacer. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as an extracellular spacer. In some embodiments, the extracellular spacer includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the extracellular spacer includes about 1 to 10, about 5 to 15, about 10 to 20, about 15 to 25, about 20 to 40, about 30 to 50, about 40 to 60, about 50 to 70 amino acid residues. In some embodiments, the extracellular spacer includes about 40 to 70, about 50 to 80, about 60 to 80, about 70 to 90, or about 80 to 100 amino acid residues. In some embodiments, the extracellular spacer includes about 1 to 10, about 5 to 15, about 10 to 20, about 15 to 25 amino acid residues. In some embodiments, the length and amino acid composition of the extracellular spacer can be optimized to vary the orientation and/or proximity of the ECD and the hinge domain to one another to achieve a desired activity of the CARs. In some embodiments, the orientation and/or proximity of the ECD and the hinge domain to one another can be varied and/or optimized as a “tuning” tool or effect that would enhance or reduce the efficacy of the CARs. In some embodiments, the orientation and/or proximity of the ECD and the hinge domain to one another can be varied and/or optimized to create fully functional or partially functional versions of the CARs. In some embodiments, the extracellular spacer domain includes an amino acid sequence corresponding to an IgG4 hinge domain and an IgG4 CH2-CH3 domain.

In some embodiments, the chimeric polypeptide includes, in N-terminal to C-terminal direction: (i) an ECD capable of binding CD19 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain. In some embodiments, the chimeric polypeptide includes, in N-terminal to C-terminal direction: (i) an ECD capable of binding CD19 antigen; (ii) a hinge domain from CD28; (iii) a TMD from CD8; (iv) an ICD including a costimulatory domain from 4-1BB; and (v) a CD3ζ domain. In some embodiments, the chimeric polypeptide includes, in N-terminal to C-terminal direction: (i) an ECD capable of binding CD19 antigen; (ii) a hinge domain from CD28; (iii) a TMD from CD8; and (iv) a CD3ζ domain.

In some embodiments, the chimeric polypeptide includes, in N-terminal to C-terminal direction: (i) an ECD capable of binding HER2 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the chimeric polypeptide includes, in N-terminal to C-terminal direction: (i) an ECD capable of binding B7-H3 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the chimeric polypeptide includes, in N-terminal to C-terminal direction: (i) an ECD capable of binding GPC2 antigen; (ii) a hinge domain from CD28; (iii) a TMD from CD8, CD28, CD3, CD4, CTLA4, or PD-1; (iii) an ICD including a costimulatory domain from 4-1BB; and (iv) a CD3ζ domain.

In some embodiments, the chimeric polypeptide has an amino acid sequence having at least 80% sequence identity to the amino acid sequence of SEQ ID NO: 13. In some embodiments, the chimeric polypeptide has an amino acid sequence having at least 80%, at least 85%, at least 90%, at least 95% sequence identity to the amino acid sequence of SEQ ID NO: 13. In some embodiments, the chimeric polypeptide has an amino acid sequence having at least 95%, at least 96%, at least 97%, at least 98%, at least 99% sequence identity to the amino acid sequence of SEQ ID NO: 13. In some embodiments, the chimeric polypeptide has an amino acid sequence having 100% sequence identity to the amino acid sequence of SEQ ID NO: 13.

In some embodiments, the chimeric polypeptide has an amino acid sequence having at least 80% sequence identity to the amino acid sequence of SEQ ID NO: 27. In some embodiments, the chimeric polypeptide has an amino acid sequence having at least 80%, at least 85%, at least 90%, at least 95% sequence identity to the amino acid sequence of SEQ ID NO: 27. In some embodiments, the chimeric polypeptide has an amino acid sequence having at least 95%, at least 96%, at least 97%, at least 98%, at least 99% sequence identity to the amino acid sequence of SEQ ID NO: 27. In some embodiments, the chimeric polypeptide has an amino acid sequence having 100% sequence identity to the amino acid sequence of SEQ ID NO: 27.

In some embodiments, the chimeric polypeptide has an amino acid sequence having at least 80% sequence identity to the amino acid sequence of SEQ ID NO: 39. In some embodiments, the chimeric polypeptide has an amino acid sequence having at least 80%, at least 85%, at least 90%, at least 95% sequence identity to the amino acid sequence of SEQ ID NO: 39. In some embodiments, the chimeric polypeptide has an amino acid sequence having at least 95%, at least 96%, at least 97%, at least 98%, at least 99% sequence identity to the amino acid sequence of SEQ ID NO: 39. In some embodiments, the chimeric polypeptide has an amino acid sequence having 100% sequence identity to the amino acid sequence of SEQ ID NO: 39.

In some embodiments, the chimeric polypeptide has an amino acid sequence having at least 80% sequence identity to the amino acid sequence of SEQ ID NO: 53. In some embodiments, the chimeric polypeptide has an amino acid sequence having at least 80%, at least 85%, at least 90%, at least 95% sequence identity to the amino acid sequence of SEQ ID NO: 53. In some embodiments, the chimeric polypeptide has an amino acid sequence having at least 95%, at least 96%, at least 97%, at least 98%, at least 99% sequence identity to the amino acid sequence of SEQ ID NO: 53. In some embodiments, the chimeric polypeptide has an amino acid sequence having 100% sequence identity to the amino acid sequence of SEQ ID NO: 53.

In some embodiments, the chimeric polypeptide has an amino acid sequence having at least 80% sequence identity to the amino acid sequence of SEQ ID NO: 67. In some embodiments, the chimeric polypeptide has an amino acid sequence having at least 80%, at least 85%, at least 90%, at least 95% sequence identity to the amino acid sequence of SEQ ID NO: 67. In some embodiments, the chimeric polypeptide has an amino acid sequence having at least 95%, at least 96%, at least 97%, at least 98%, at least 99% sequence identity to the amino acid sequence of SEQ ID NO: 67. In some embodiments, the chimeric polypeptide has an amino acid sequence having 100% sequence identity to the amino acid sequence of SEQ ID NO: 67.

One skilled in the art will appreciate that the complete amino acid sequence of a chimeric polypeptide or CAR of the disclosure can be used to construct a back-translated gene. For example, a DNA oligomer containing a nucleotide sequence coding for a given chimeric polypeptide or CAR can be synthesized. For example, several small oligonucleotides coding for portions of the desired CAR or antibody can be synthesized and then ligated. The individual oligonucleotides typically contain 5′ or 3′ overhangs for complementary assembly.

In addition to generating desired chimeric polypeptides or CARs via expression of nucleic acid molecules that have been altered by recombinant molecular biological techniques, a subject chimeric polypeptide or CAR in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a chimeric polypeptide or CAR as disclosed herein can be inserted into an expression vector and operably linked to an expression control sequence appropriate for expression of the chimeric polypeptide or CAR in the desired transformed host. Proper assembly can be confirmed by nucleotide sequencing, restriction mapping, and expression of a biologically active polypeptide in a suitable host. As is known in the art, in order to obtain high expression levels of a transfected gene in a host, take should be taken to ensure that the gene is operably linked to transcriptional and translational expression control sequences that are functional in the chosen expression host.

### Nucleic Acid Molecules

In one aspect, provided herein are various nucleic acid molecules including nucleotide sequences encoding a chimeric polypeptide of the disclosure, including expression cassettes, and expression vectors containing these nucleic acid molecules operably linked to heterologous nucleic acid sequences such as, for example, regulator sequences which allow in vivo expression of the chimeric polypeptide in a host cell or ex-vivo cell-free expression system.

Nucleic acid molecules of the present disclosure can be nucleic acid molecules of any length, including nucleic acid molecules that are generally between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the recombinant nucleic acid includes a nucleic acid sequence encoding a CAR that includes (i) a first polypeptide segment including an ECD capable of binding an antigen; (ii) a second polypeptide segment including a hinge domain from CD28; (iii) a third polypeptide segment including a TMD. In some embodiments, the CAR encoded by the nucleic acid sequence further includes a fourth polypeptide segment including an ICD including a costimulatory domain, wherein the costimulatory domain is not from CD28.

In some embodiments, the recombinant nucleic acid includes a nucleic acid sequence encoding a CAR that includes, in N-terminal to C-terminal direction: (i) an ECD capable of binding CD19 antigen; (ii) a hinge domain from CD28; (iii) a TMD from CD8, CD28, CD3, CD4, CTLA4, or PD-1. In some embodiments, the CAR encoded by the nucleic acid sequence further includes an ICD including (iv) a costimulatory domain from 4-1BB and/or (v) a CD3ζ domain.

In some embodiments, the recombinant nucleic acid includes a nucleic acid sequence encoding a CAR that includes, in N-terminal to C-terminal direction: (i) an ECD capable of binding CD19 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain. In some embodiments, the recombinant nucleic acid includes a nucleic acid sequence encoding a CAR that includes, in N-terminal to C-terminal direction: (i) an ECD capable of binding CD19 antigen; (ii) a hinge domain from CD28; (iii) a TMD from CD8; (iv) an ICD including a costimulatory domain from 4-1BB; and (v) a CD3ζ domain.

In some embodiments, the recombinant nucleic acid includes a nucleic acid sequence encoding a CAR that includes, in N-terminal to C-terminal direction: (i) an ECD capable of binding CD19 antigen; (ii) a hinge domain from CD28; (iii) a TMD from CD8; and (iv) a CD3ζ domain.

In some embodiments, the recombinant nucleic acid includes a nucleic acid sequence encoding a CAR that includes, in N-terminal to C-terminal direction: (i) an ECD capable of binding HER2 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant nucleic acid includes a nucleic acid sequence encoding a CAR that includes, in N-terminal to C-terminal direction: (i) an ECD capable of binding B7-H3 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant nucleic acid includes a nucleic acid sequence encoding a CAR that includes, in N-terminal to C-terminal direction: (i) an ECD capable of binding GPC2 antigen; (ii) a hinge domain from CD28; (iii) a TMD from CD8, CD28, CD3, CD4, CTLA4, or PD-1; (iii) an ICD including a costimulatory domain from 4-1BB; and (iv) a CD3ζ domain.

In some embodiments, the recombinant nucleic acid includes a nucleic acid sequence having at least 80% sequence identity to a nucleic acid sequence selected from the group consisting of SEQ ID NO: 14, SEQ ID NO: 28, SEQ ID NO: 40, SEQ ID NO: 54, and SEQ ID NO: 68. In some embodiments, the recombinant nucleic acid includes a nucleic acid sequence having at least 85%, at least 90%, at least 95%, at least 96%, at least 97%, at least 98%, at least 99%, or at least 100% sequence identity sequence identity to a nucleic acid sequence selected from the group consisting of SEQ ID NO: 14, SEQ ID NO: 28, SEQ ID NO: 40, SEQ ID NO: 54, and SEQ ID NO: 68.

In some embodiments, the recombinant nucleic acid includes a nucleic acid sequence having at least 80% sequence identity to the nucleic acid sequence of SEQ ID NO: 14. In some embodiments, the recombinant nucleic acid includes a nucleic acid sequence having at least 80%, at least 85%, at least 90%, at least 95% sequence identity to the nucleic acid sequence of SEQ ID NO: 14. In some embodiments, the recombinant nucleic acid includes a nucleic acid sequence having at least 95%, at least 96%, at least 97%, at least 98%, at least 99% sequence identity to the nucleic acid sequence of SEQ ID NO: 14. In some embodiments, the recombinant nucleic acid includes a nucleic acid sequence having 100% sequence identity to the nucleic acid sequence of SEQ ID NO: 14.

In some embodiments, the recombinant nucleic acid includes a nucleic acid sequence having at least 80% sequence identity to the nucleic acid sequence of SEQ ID NO: 28. In some embodiments, the recombinant nucleic acid includes a nucleic acid sequence having at least 80%, at least 85%, at least 90%, at least 95% sequence identity to the nucleic acid sequence of SEQ ID NO: 28. In some embodiments, the recombinant nucleic acid includes a nucleic acid sequence having at least 95%, at least 96%, at least 97%, at least 98%, at least 99% sequence identity to the nucleic acid sequence of SEQ ID NO: 28. In some embodiments, the recombinant nucleic acid includes a nucleic acid sequence having 100% sequence identity to the nucleic acid sequence of SEQ ID NO: 28.

In some embodiments, the recombinant nucleic acid includes a nucleic acid sequence having at least 80% sequence identity to the nucleic acid sequence of SEQ ID NO: 40. In some embodiments, the recombinant nucleic acid includes a nucleic acid sequence having at least 80%, at least 85%, at least 90%, at least 95% sequence identity to the nucleic acid sequence of SEQ ID NO: 40. In some embodiments, the recombinant nucleic acid includes a nucleic acid sequence having at least 95%, at least 96%, at least 97%, at least 98%, at least 99% sequence identity to the nucleic acid sequence of SEQ ID NO: 40. In some embodiments, the recombinant nucleic acid includes a nucleic acid sequence having 100% sequence identity to the nucleic acid sequence of SEQ ID NO: 40.

In some embodiments, the recombinant nucleic acid includes a nucleic acid sequence having at least 80% sequence identity to the nucleic acid sequence of SEQ ID NO: 54. In some embodiments, the recombinant nucleic acid includes a nucleic acid sequence having at least 80%, at least 85%, at least 90%, at least 95% sequence identity to the nucleic acid sequence of SEQ ID NO: 54. In some embodiments, the recombinant nucleic acid includes a nucleic acid sequence having at least 95%, at least 96%, at least 97%, at least 98%, at least 99% sequence identity to the nucleic acid sequence of SEQ ID NO: 54. In some embodiments, the recombinant nucleic acid includes a nucleic acid sequence having 100% sequence identity to the nucleic acid sequence of SEQ ID NO: 54.

In some embodiments, the recombinant nucleic acid includes a nucleic acid sequence having at least 80% sequence identity to the nucleic acid sequence of SEQ ID NO: 68. In some embodiments, the recombinant nucleic acid includes a nucleic acid sequence having at least 80%, at least 85%, at least 90%, at least 95% sequence identity to the nucleic acid sequence of SEQ ID NO: 68. In some embodiments, the recombinant nucleic acid includes a nucleic acid sequence having at least 95%, at least 96%, at least 97%, at least 98%, at least 99% sequence identity to the nucleic acid sequence of SEQ ID NO: 68. In some embodiments, the recombinant nucleic acid includes a nucleic acid sequence having 100% sequence identity to the nucleic acid sequence of SEQ ID NO: 68.

In some embodiments, the recombinant nucleic acid molecule is operably linked to a heterologous nucleic acid sequence.

In some embodiments, the recombinant nucleic acid molecule is further defined as an expression cassette or a vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric polypeptide as disclosed herein, which is operably linked to expression control elements, such as a promoter, and optionally, any other sequences or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 80%, 90%, 95%, 96%, 97, 98%, 99%, or 100% sequence identity to a chimeric polypeptide disclosed herein. In some embodiments, provided herein are nucleic acid molecules encoding a polypeptide with an amino acid sequence having at least about 80% sequence identity to any one of SEQ ID NO: 13, SEQ ID NO: 27, SEQ ID NO: 39, SEQ ID NO: 53, and, SEQ ID NO: 67. In some embodiments, the nucleic acid molecules encode a polypeptide with an amino acid sequence having at least about 80%, 90%, 95%, 96%, 97, 98%, 99%, or 100% sequence identity to SEQ ID NO: 13. In some embodiments, the nucleic acid molecules encode a polypeptide with an amino acid sequence having at least about 80%, 90%, 95%, 96%, 97, 98%, 99%, or 100% sequence identity to SEQ ID NO: 27. In some embodiments, the nucleic acid molecules encode a polypeptide with an amino acid sequence having at least about 80%, 90%, 95%, 96%, 97, 98%, 99%, or 100% sequence identity to SEQ ID NO: 39. In some embodiments, the nucleic acid molecules encode a polypeptide with an amino acid sequence having at least about 80%, 90%, 95%, 96%, 97, 98%, 99%, or 100% sequence identity to SEQ ID NO: 53. In some embodiments, the nucleic acid molecules encode a polypeptide with an amino acid sequence having at least about 80%, 90%, 95%, 96%, 97, 98%, 99%, or 100% sequence identity to SEQ ID NO: 67.

The nucleic acid sequences encoding the chimeric polypeptides can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an anti sense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid molecules of the present disclosure can be introduced into a cell, such as a human T cell or cancer cell, to produce a recombinant cell containing the nucleic acid molecule. Accordingly, some embodiments of the disclosure relate to methods for making a recombinant cell, including (a) providing a host cell capable of protein expression; and transducing the provided host cell with a recombinant nucleic acid of the disclosure to produce a recombinant cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be introduced into a host cell by viral or non-viral delivery vehicles known in the art to produce an engineered cell. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using zinc-finger proteins (ZNF), guide RNA directed CRISPR/Cas9, DNA-guided endonuclease genome editing NgAgo (Natronobacterium gregoryi Argonaute), or TALEN genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the chimeric polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a mouse cell. In some embodiments, the animal cell is a human cell. In some embodiments, the cell is a non-human primate cell. In some embodiments, the recombinant cell is an immune system cell, e.g., a B cell, a monocyte, a NK cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor.

In some embodiments, the immune system cell is a lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+ T cytotoxic lymphocyte cell. Non-limiting examples of CD8+ T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell. Suitable CD4+ T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells.

As outlined above, some embodiments of the disclosure relate to various methods for making a recombinant cell, including (a) providing a host cell capable of protein expression; and transducing the provided host cell with a recombinant nucleic acid of the disclosure to produce a recombinant cell. Non-limiting exemplary embodiments of the disclosed methods for making a recombinant cell can further include one or more of the following features. In some embodiments, the host cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the recombinant cells produced by the methods disclosed herein are also within the scope of the disclosure.

Techniques for transforming a wide variety of the above-mentioned host cells and species are known in the art and described in the technical and scientific literature. For example, DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application. Methods and systems suitable for generating and maintaining cell cultures are known in the art.

In one aspect, some embodiments of the disclosure relate to a recombinant cell including: (a) a chimeric polypeptide as described herein; and/or a nucleic acid molecule according as described herein. In some embodiments, the recombinant cell of the disclosure includes a nucleic acid molecule encoding a CAR that includes (i) a first polypeptide segment including an ECD capable of binding an antigen; (ii) a second polypeptide segment including a hinge domain from CD28; (iii) a third polypeptide segment including a TMD. In some embodiments, the CAR encoded by the nucleic acid sequence further includes (iv) a fourth polypeptide segment including an ICD including a costimulatory domain, wherein the costimulatory domain is not from CD28.

In some embodiments, the recombinant cell includes a nucleic acid molecule encoding a CAR that includes, in N-terminal to C-terminal direction: (i) an ECD capable of binding CD19 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant cell includes a nucleic acid molecule encoding a CAR that includes, in N-terminal to C-terminal direction: (i) an ECD capable of binding CD19 antigen; (ii) a hinge domain from CD28; (iii) a TMD from CD8; (iv) an ICD including a costimulatory domain from 4-1BB; and (v) a CD3ζ domain.

In some embodiments, the recombinant cell includes a nucleic acid molecule encoding a CAR that includes, in N-terminal to C-terminal direction: (i) an ECD capable of binding CD19 antigen; (ii) a hinge domain from CD28; (iii) a TMD from CD8; and (iv) a CD3ζ domain.

In some embodiments, the recombinant cell includes a nucleic acid molecule encoding a CAR that includes, in N-terminal to C-terminal direction: (i) an ECD capable of binding HER2 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant cell includes a nucleic acid molecule encoding a CAR that includes, in N-terminal to C-terminal direction: (i) an ECD capable of binding B7-H3 antigen; (ii) a hinge domain from CD28; (iii) a TMD from CD8, CD28, CD3, CD4, CTLA4, or PD-1; (iv) an ICD including a costimulatory domain from 4-1BB; and (v) a CD3ζ domain.

In some embodiments, the recombinant cell includes a nucleic acid molecule encoding a CAR that includes, in N-terminal to C-terminal direction: (i) an ECD capable of binding GPC2 antigen; (ii) a hinge domain from CD28; (iii) a TMD from CD8, CD28, CD3, CD4, CTLA4, or PD-1; (iii) an ICD including a costimulatory domain from 4-1BB; and (iv) a CD3ζ domain.

In some embodiments, the recombinant cell includes a nucleic acid molecule including a nucleic acid sequence encoding a CAR which at least 80% sequence identity to an amino acid sequence selected from the group consisting of SEQ ID NO: 13. In some embodiments, the recombinant cell includes a nucleic acid molecule including a nucleic acid sequence encoding a CAR which at least 80% sequence identity to an amino acid sequence selected from the group consisting of SEQ ID NO: 27. In some embodiments, the recombinant cell includes a nucleic acid molecule including a nucleic acid sequence encoding a CAR which at least 80% sequence identity to an amino acid sequence selected from the group consisting of SEQ ID NO: 39. In some embodiments, the recombinant cell includes a nucleic acid molecule including a nucleic acid sequence encoding a CAR which at least 80% sequence identity to an amino acid sequence selected from the group consisting of SEQ ID NO: 53. In some embodiments, the recombinant cell includes a nucleic acid molecule including a nucleic acid sequence encoding a CAR which at least 80% sequence identity to an amino acid sequence selected from the group consisting of SEQ ID NO: 67.

In a related aspect, some embodiments of the disclosure relate to cell cultures including at least one recombinant cell as disclosed herein, and a culture medium. Generally, the culture medium can be any one of suitable culture media for the cell cultures described herein. In some embodiments, the recombinant cell expresses a chimeric polypeptide or a CAR described herein. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the chimeric polypeptides, chimeric antigen receptors (CARs), nucleic acids, recombinant cells, and/or cell cultures of the disclosure can be incorporated into compositions, including pharmaceutical compositions. Such compositions generally include the chimeric polypeptides, CARs, nucleic acids, recombinant cells, and/or cell cultures as described herein and a pharmaceutically acceptable carrier. Accordingly, in one aspect, some embodiments of the disclosure relate to pharmaceutical compositions for treating, preventing, ameliorating, reducing or delaying the onset of a health condition, for example a proliferative disease (e.g., cancer).

Accordingly, one aspect of the present disclosure relates to pharmaceutical compositions that include a pharmaceutically acceptable carrier and one or more of the following: (a) a chimeric polypeptide of the disclosure; (b) a nucleic acid molecule of the disclosure; and/or (c) a recombinant cell of the disclosure. In some embodiments, the composition includes (a) a recombinant nucleic acid of the disclosure and (b) a pharmaceutically acceptable carrier. In some embodiments, the recombinant nucleic acid is encapsulated in a viral capsid or a lipid nanoparticle. In some embodiments, the composition includes (a) a recombinant cell of the disclosure and (b) a pharmaceutically acceptable carrier.

In certain embodiments, the pharmaceutical compositions in accordance with some embodiments disclosed herein include cell cultures that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Methods of Treatment

Administration of any one of the therapeutic compositions described herein, e.g., chimeric polypeptides, CARs, nucleic acids, recombinant cells, cell cultures, and/or pharmaceutical compositions, can be used in the diagnosis, prevention, and/or treatment of relevant conditions, such as proliferative diseases (e.g., cancer). In some embodiments, the chimeric polypeptides, CARs, nucleic acids, recombinant cells, cell cultures, and/or pharmaceutical compositions as described herein can be incorporated into therapies and therapeutic agents for use in methods of preventing and/or treating an individual who has, who is suspected of having, or who may be at high risk for developing one or more health conditions, such as proliferative diseases (e.g., cancers). In some embodiments, the individual is a patient under the care of a physician.

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Accordingly, in one aspect, some embodiments of the disclosure relate to methods for the prevention and/or treatment of a condition in a subject in need thereof, wherein the methods include administering to the subject a composition including one or more of: a chimeric polypeptide of the disclosure, a recombinant nucleic acid of the disclosure, a recombinant cell of the disclosure, and/or a pharmaceutical composition of the disclosure.

In some embodiments, the compositions described herein, e.g., polypeptides, CARs, nucleic acids, recombinant cells, cell cultures, and/or pharmaceutical compositions, can be used in methods of treating individual who have, who are suspected of having, or who may be at high risk for developing leukemia. In these instances, the leukemia can generally be of any type of leukemia. Suitable leukemia that can be treated using the compositions described herein (e.g., polypeptides, CARs, nucleic acids, recombinant cells, cell cultures, and/or pharmaceutical compositions) include, but are not limited to, acute lymphoblastic leukemia (ALL), acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), and hairy cell leukemia. In some embodiments, the leukemia is AML.

In some embodiments, the administered composition confers increased production of interferon gamma (IFNγ) and/or interleukin-2 (IL-2) in the subject compared with a reference subject that has not been administered with the same composition.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit the proliferation of the target cell and/or inhibit tumor growth of a target cancer in the subject compared to the proliferation of the target cell and/or tumor growth of the target cancer in subjects who have not been administered with the recombinant cells.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

Administration of the compositions described herein, e.g., polypeptides, CARs, nucleic acids, recombinant cells, cell cultures, and/or pharmaceutical compositions, can be used in the stimulation of an immune response. In some embodiments, polypeptides, CARs, nucleic acids, recombinant cells, cell cultures, and/or pharmaceutical compositions as described herein are administered to an individual after induction of remission of cancer with chemotherapy, or after autologous or allogeneic hematopoietic stem cell transplantation. In some embodiments, compositions described herein are administered to an individual in need of increasing the production of interferon gamma (IFNγ) and/or interleukin-2 (IL-2) in the treated subject relative to the production of these molecules in subjects who have not been administered one of the therapeutic compositions disclosed herein.

An effective amount of the compositions described herein, e.g., polypeptides, CARs, nucleic acids, recombinant cells, cell cultures, and/or pharmaceutical compositions, is determined based on the intended goal, for example tumor regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each individual. Frequency of administration could range from 1-2 days, to 2-6 hours, to 6-10 hours, to 1-2 weeks or longer depending on the judgment of the practitioner.

Longer intervals between administration and lower amounts of compositions may be employed where the goal is prevention. For instance, amounts of compositions administered per dose may be 50% of the dose administered in treatment of active disease, and administration may be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In certain embodiments, it may be desirable to provide a continuous supply of a composition disclosed herein to the subject to be treated, e.g., a patient. In some embodiments, continuous perfusion of the region of interest (such as the tumor) may be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by bolus injection. In some embodiments, administration is by intravenous infusion. In some embodiments, a composition is administered is administered in a dosage of about 100 ng/kg of body weight per day to about 100 mg/kg of body weight per day. In some embodiments, a composition as disclosed herein is administered in a dosage of about 0.001 mg/kg to 100 mg/kg of body weight per day. In some embodiments, the therapeutic agents are administered in a single administration. In some embodiments, therapeutic agents are administered in multiple administrations, (e.g., once or more per week for one or more weeks). In some embodiments, doses are administered about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days. In some embodiments, there are 1, 2, 3, 4, 5, 6, 7, 8, 9, 10, or more total doses. In some embodiments, 4 doses are administered, with a 3 week span between doses.

One of ordinary skill in the art would be familiar with techniques for administering compositions of the disclosure to an individual. Furthermore, one of ordinary skill in the art would be familiar with techniques and pharmaceutical reagents necessary for preparation of these compositions prior to administration to an individual.

In certain embodiments of the present disclosure, the composition of the disclosure will be an aqueous composition that includes one or more of the chimeric polypeptides, CARs, nucleic acids, recombinant cells, cell cultures, and/or pharmaceutical compositions as described herein. Aqueous compositions of the present disclosure contain an effective amount of a composition disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

One of ordinary skill in the art would appreciate that biological materials should be extensively dialyzed to remove undesired small molecular weight molecules and/or lyophilized for more ready formulation into a desired vehicle, where appropriate. The compositions described herein, e.g., polypeptides, CARs, nucleic acids, recombinant cells, cell cultures, and/or pharmaceutical compositions, will then generally be formulated for administration by any known route, such as parenteral administration. Determination of the amount of compositions to be administered will be made by one of skill in the art, and will in part be dependent on the extent and severity of cancer, and whether the recombinant cells are being administered for treatment of existing cancer or prevention of cancer. The preparation of the compositions containing the chimeric polypeptides, CARs, nucleic acids, recombinant cells, cell cultures, and/or pharmaceutical compositions of the disclosure will be known to those of skill in the art in light of the present disclosure.

Upon formulation, the compositions of the disclosure will be administered in a manner compatible with the dosage formulation and in such amount as is therapeutically effective. The compositions can be administered in a variety of dosage forms, such as the type of injectable solutions described above. For parenteral administration, the compositions disclosed herein should be suitably buffered. As discussed in greater detail below, the compositions as described herein may be administered with other therapeutic agents that are part of the therapeutic regiment of the individual, such as other immunotherapy or chemotherapy. The chimeric polypeptides, CARs, nucleic acids, recombinant cells, cell cultures, and/or pharmaceutical compositions described herein can be used to inhibit tumor growth or metastasis of a cancer in the treated subject relative to the tumor growth or metastasis in subjects who have not been administered one of the therapeutic compositions disclosed herein. In some embodiments, the antibodies, CARs, nucleic acids, recombinant cells, cell cultures, and/or pharmaceutical compositions described herein can be used to stimulate immune responses against the tumor via inducing the production of interferon gamma (IFNγ) and/or interleukin-2 (IL-2) and other pro-inflammatory cytokines. In some embodiments, the antibodies, CARs, nucleic acids, recombinant cells, cell cultures, and/or pharmaceutical compositions described herein can be used to stimulate proliferation and/or killing capacity of CAR T-cells in the treated subject relative to the production of these molecules in subjects who have not been administered one of the therapeutic compositions disclosed herein. The production of interferon gamma (IFNγ) and/or interleukin-2 (IL-2) can be stimulated to produce up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the production of interferon gamma (IFNγ) and/or interleukin-2 (IL-2) in subjects who have not been administered one of the therapeutic compositions disclosed herein.

### Administration of Recombinant Cells to a Subject

In some embodiments, the methods of the disclosure involve administering an effective amount or number of the recombinants cells provided here to a subject in need thereof. This administering step can be accomplished using any method of implantation delivery in the art. For example, the recombinant cells can be infused directly in the subject's bloodstream or otherwise administered to the subject.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, i.e., long-term engraftment.

When provided prophylactically, the recombinant cells described herein can be administered to a subject in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to a subject in need thereof.

In some embodiments, the delivery of a recombinant cell composition (e.g., a composition including a plurality of recombinant cells according to any of the cells described herein) into a subject by a method or route results in at least partial localization of the cell composition at a desired site. A composition including recombinant cells can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection. For example, a population of recombinant cells are administered other than directly into a target site, tissue, or organ, such that it enters, the subject's circulatory system and, thus, is subject to metabolism and other similar biological processes.

The efficacy of a treatment including any of the compositions provided herein for the prevention or treatment of a disease or condition can be determined by a skilled clinician. However, one skilled in the art will appreciate that a prevention or treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99, or 100%.

As discussed above, a therapeutically effective amount includes an amount of a therapeutic composition that is sufficient to promote a particular beneficial effect when administered to a subject, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

### Additional Therapies

As discussed above, any one of the compositions as disclosed herein, e.g., chimeric polypeptides, CARs, nucleic acids, recombinant cells, cell cultures, and/or pharmaceutical compositions, can be administered to a subject in need thereof as a single therapy (e.g., monotherapy). In addition or alternatively, in some embodiments of the disclosure, the chimeric polypeptides, CARs, nucleic acids, recombinant cells, cell cultures, and/or pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of prevention and/or treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein. In particular, some embodiments of the disclosure provide kits for the diagnosis of a condition in a subject. Some other embodiments relate to kits for the prevention of a condition in a subject in need thereof. Some other embodiments relate to kits for methods of treating a condition in a subject in need thereof. For example, provided herein, in some embodiments, are kits that include one or more of the chimeric polypeptides, recombinant nucleic acids, engineered cells, or pharmaceutical compositions as provided and described herein, as well as written instructions for making and using the same.

In some embodiments, the kits of the disclosure further include one or more means useful for the administration of any one of the provided chimeric polypeptides, recombinant nucleic acids, engineered cells, or pharmaceutical compositions to an individual. For example, in some embodiments, the kits of the disclosure further include one or more syringes (including pre-filled syringes) and/or catheters (including pre-filled syringes) used to administer any one of the provided chimeric polypeptides, recombinant nucleic acids, engineered cells, or pharmaceutical compositions to an individual. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative control polypeptides, positive control polypeptides, reagents suitable for in vitro production of the chimeric polypeptides.

In some embodiments, the components of a kit can be in separate containers. In some other embodiments, the components of a kit can be combined in a single container.

In some embodiments, a kit can further include instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

### Integration of a CD28 Hinge into a CD19 CAR (CD19-28Hinge-28TM-41BBz) Resulted in Enhancement of Killing CD19^{low} Cells and Cytokine Production

This Example describes experiments performed to demonstrate that incorporation of the CD28 hinge into a CD19 CAR (CD19-28Hinge-28TM-41BBz) resulted in enhancement of killing CD19low cells and cytokine production in response to a range of CD19 antigen densities compared to CD19-CD8Hinge-CD8TM-41BBz (Kymriah), comparing favorably to a CD19-28z CAR (Axi-Cel).

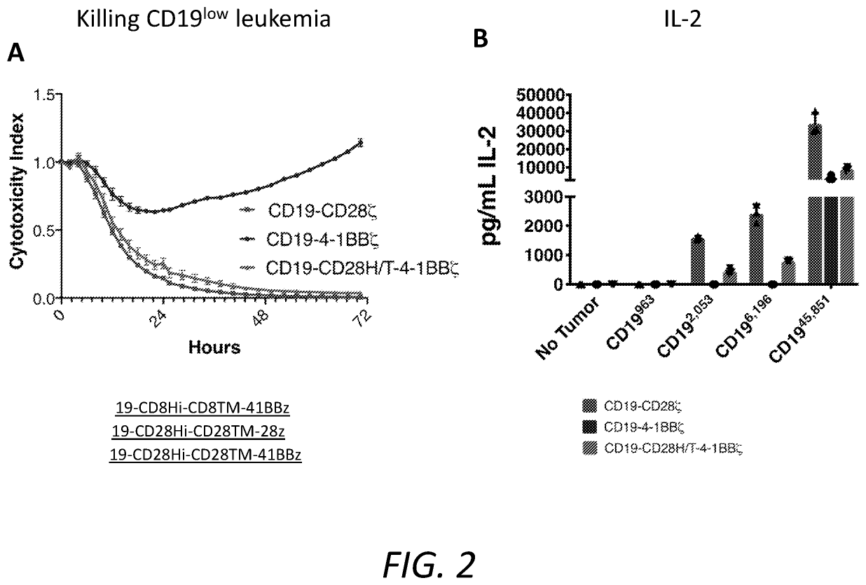

As shown in FIG. 2A, retroviral vectors encoding CD19 CARs with the indicated structures were synthesized commercially and cloned by standard methods. Viral supernatant was produced in 293GP cells after transient transfection of the retroviral plasmid. NALM6^{low} cells were generated by using a CRISPR-Cas9 technique to knockout CD19 from the NALM6 tumor line and then reintroducing a truncated version of the protein (extracellular and transmembrane portions only) using a lentivirus-based vector. Cells were FACS sorted and single-cell cloned to achieve a library of clones of different CD19 antigen densities. CD19 CARs were transduced into human T cells. Primary human T cells were transduced with viral supernatant after activation with CD3/CD28 beads. The CD19 CARs with the indicated structures were co-cultured with NALM6 cells expressing very low levels of CD19 (approximately 1,000 molecules per cell) and tumor cells remaining (survival) were measured over time in an Incucyte by measuring GFP (the NALM6 cells express GFP). As shown in FIG. 2A, NALM6 clones expressing 963 molecules of surface CD19 were co-cultured at a 1:1 ratio with either CD19-CD28ζ, CD19-4-1BBζ, or CD19-CD28H/T-4-1BBζ CAR T cells and tumor cell killing was measured in an Incucyte assay. Representative of three experiments with different T cell donors. Statistical analysis performed with repeated measures ANOVA. It was observed that the inclusion of the CD28 hinge and CD28 TMDs in a CD19 CAR containing the 4-1BB and CD3-zeta endodomains resulted in enhanced cytolytic function against tumor with low antigen density compared to a traditional CD19-41BB-zeta CAR, similarly to a traditional CD19-CD28-zeta CAR. It was observed that the inclusion of the CD28 hinge and CD28 TMDs in a CD19 CAR containing the 4-1BB and CD3-zeta endodomains resulted in enhanced function against tumor with low antigen density compared to a traditional CD19-41BB-zeta CAR, similarly to a traditional CD19-CD28-zeta CAR.

Additional experiments were performed to illustrate that CD19 CARs containing a 4-1BB costimulatory domain demonstrated enhanced recognition of low antigen density only when they contained a CD28 hinge domain. As shown in FIG. 2B, CD19-CD28ζ, CD19-4-1BBζ, or CD19-CD28H/T-4-1BBζ CAR T cells were co-cultured with NALM6 clones expressing various amounts of CD19 for 24 hours and IL-2 was measured in the supernatant by ELISA. Representative of three experiments with different T cell donors. Statistical comparisons performed by the student's t-test (two sided) between CD19-4-1BBζ and CD19-CD28H/T-4-1BBζ CART cells.

### Example 2

### CD19-CD28Hi-CD28TM-41BBz has Better Functionality Compared to CD19-CD8Hi-CD8TM-41BBz

This Example describes experiments performed to demonstrate that CD19-CD28Hi-CD28TM-41BBz possessed better CAR functionality compared to CD19-CD8Hi-CD8TM-41BBz for low antigen density as determined using in vivo model of CD19-low leukemia.

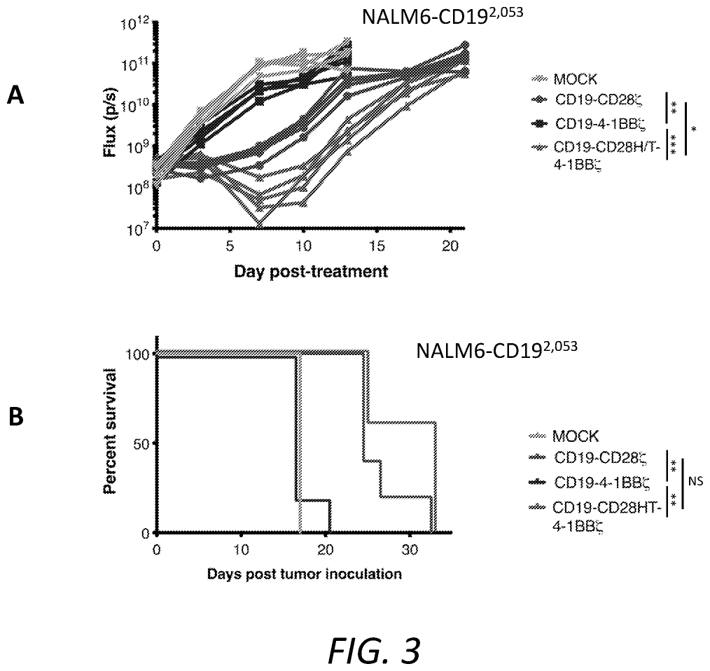

In these experiments, as shown in FIG. 3A, one million NALM6-CD^{192,053} cells were engrafted into NSG mice by tail vein injection. Four days later, mice were injected with 3 million CD19-CD28ζ, CD19-4-1BBζ, or CD19-CD28H/T-4-1BBζ CAR T cells. Tumor progression was measured by bioluminescence photometry and flux values (photons per second) were calculated using Living Image software. Quantified tumor flux values for individual mice are shown. Statistical analysis performed with repeated measures ANOVA. FIG. 3B: Mouse survival curves for mice as treated in FIG. 3A. Statistical analysis performed with the log-rank test. The results presented in FIGS. 3A-3B are representative of three experiments with different T cell donors (n=5 mice per group).

### Example 3

### CD19-CD28Hi-CD28TM-41BBz Confers Better Functionality Compared to CD19-CD8Hi-CD8TM-41BBz in Native Antigen Density

This Example describes experiments performed to demonstrate that CD19-CD28Hi-CD28TM-41BBz possessed better functionality compared to CD19-CD8Hi-CD8TM-41BBz in normal (native) antigen density, as determined by an in vivo stress test model.

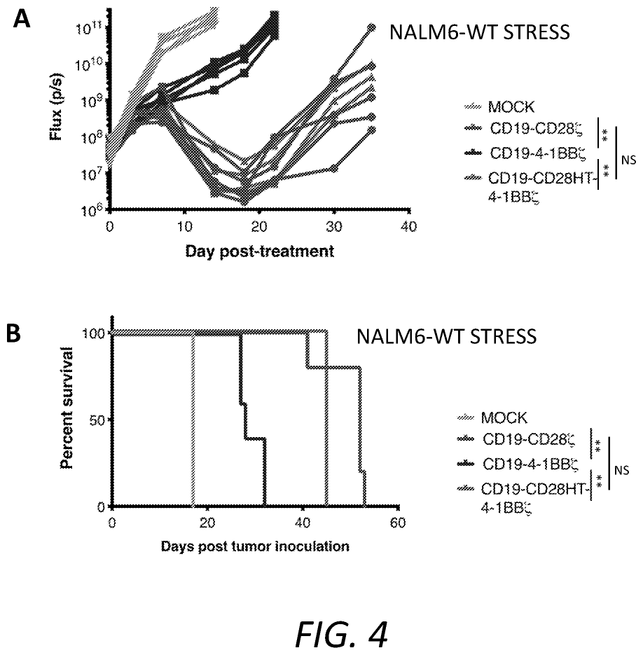

In these experiments, as shown in FIG. 4A, One million NALM6-wild-type cells were engrafted into NSG mice by tail vein injection. Three days later, mice were injected with 2.5×10⁵ CD19-CD28ζ, CD19-4-1BBζ, or CD19-CD28H/T-4-1BBζ CART cells. Tumor progression was measured by bioluminescence photometry and flux values (photons per second) were calculated using Living Image software. Quantified tumor flux values for individual mice are shown. Statistical analysis performed with repeated measures ANOVA. FIG. 4B: Mouse survival curves for mice as treated in (f). Statistical analysis performed with the log-rank test. The results presented in FIGS. 4A-4B are representative of two experiments with different T cell donors (n=5 mice per group).

### Example 4

### CD19-CD28Hi-CD28TM-41BBz Confers Better Enhanced Persistence Compared to CD19-CD28Hi-CD28TM-28z Similar to CD19-CD8Hi-CD8TM-41BB

This Example describes experiments performed to demonstrate that CD19-CD28Hi-CD28TM-41BBz endows T cells with better persistence than a CD19-CD28Hi-CD28TM-CD28z CAR as determined by flow cytometry on bone marrow and spleen samples from an in vivo Nalm6 experiment.

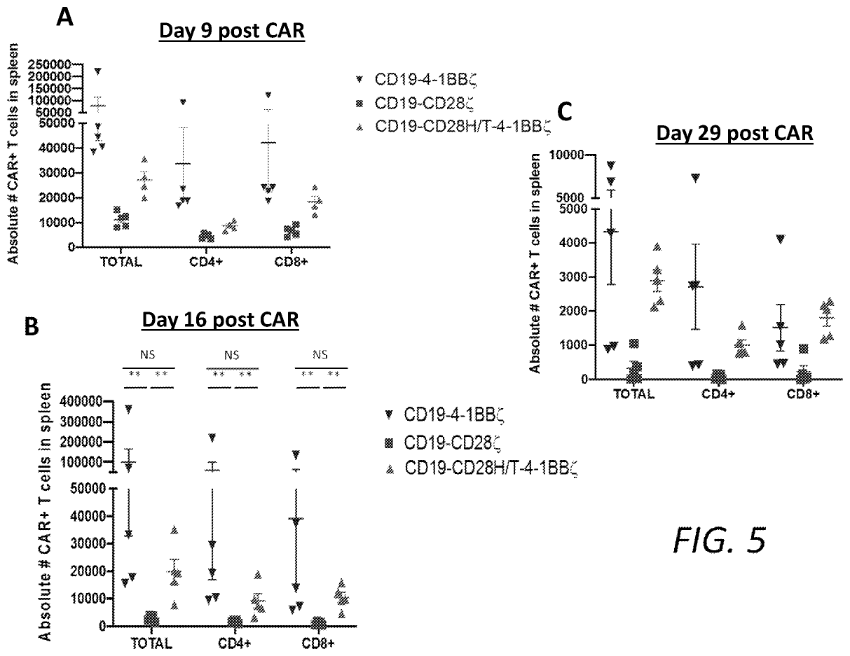

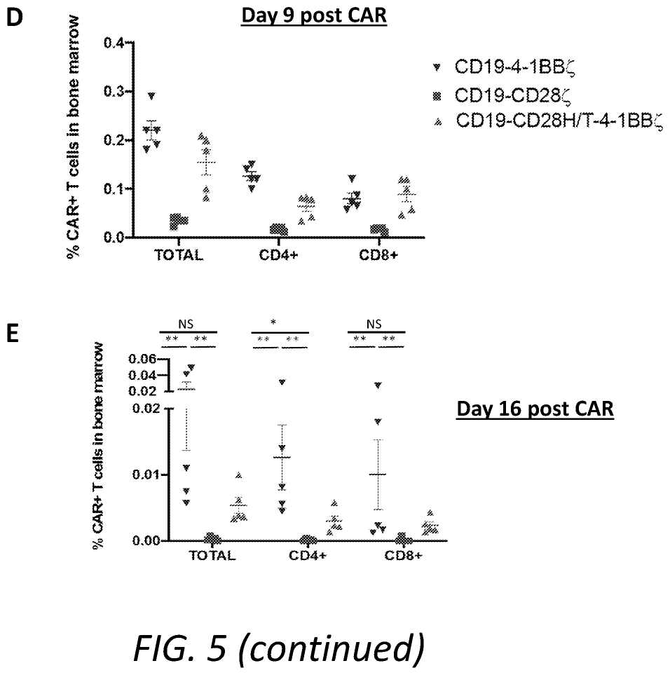

FIGS. 5A-5E schematically summarize the results of experiments performed to assess persistence of CARs targeting CD19 in spleen and bone marrow tissues. One million NALM6-wild-type cells were engrafted into NSG mice by tail vein injection. Three days later, mice were injected with 5 million CD19-CD28ζ, CD19-4-1BBζ, or CD19-CD28H/T-4-1BBζ CAR T cells. The spleens (FIGS. 5A-5C) and bone marrow (FIGS. 5D-5E) of treated mice (n=5 per group) were obtained at Day +9, +16, and +29 (post CAR T cell treatment. Presence of CAR positive T cells was assessed by flow cytometry. Performed one time (n=5 per CAR construct per timepoint). Statistical comparisons performed by Mann Whitney between the indicated groups. For in vitro experiments, error bars represent SD and for in vivo experiments, error bars represent SEM. p<0.05 was considered statistically significant, and p values are denoted with asterisks as follows: p>0.05, not significant, NS; * p<0.05, ** p<0.01, *** p<0.001, and **** p<0.0001.

### Example 5

### CD28Hi-CD28TM Confers Enhanced Reactivity in Several Tumor Models and CAR Architectures

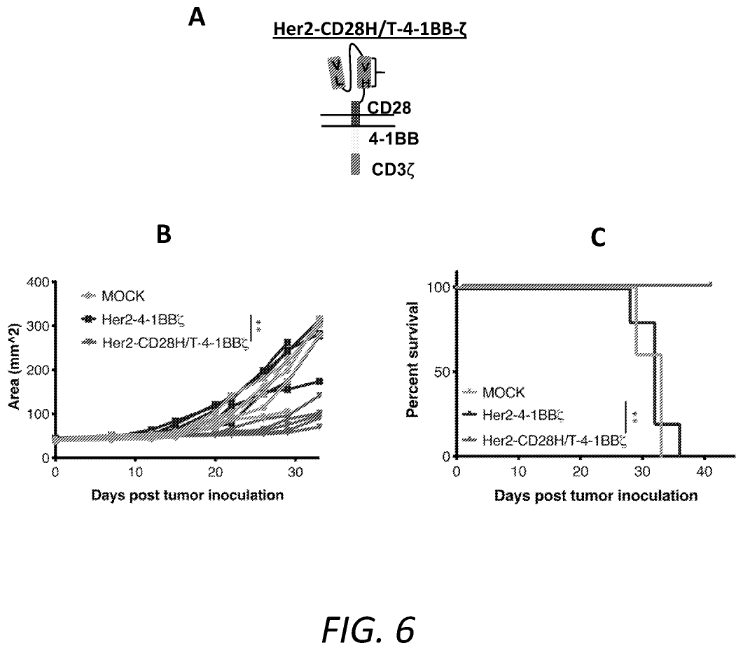

FIGS. 6A-6C schematically summarize the results of experiments performed to assess functionality of CARs targeting Her2 in a variety of tumor models and CAR architectures. FIG. 6A is a schematic of a Her2 CAR containing a CD28 hinge-transmembrane region and 4-1BB costimulatory domain (Her2-CD28H/T-4-1BBζ). FIG. 6B: One million 143b osteosarcoma cells were orthotopically implanted in the hind leg of NSG mice. After seven days, mice were treated with 10 million Her2-4-1BBζ CAR T cells, Her2-CD28H/T-4-1BBζ CAR T cells, or untransduced control T cells (MOCK). Leg measurements were obtained twice weekly with digital calibers. Measurements for individual mice are shown. Statistical analysis performed with repeated measures ANOVA. FIG. 6C: Survival curves for mice treated as in FIG. 6B: Statistical analysis performed with the log-rank test. The results presented in FIGS. 6B-6C are representative of two experiments with different T cell donors (n=5 mice per group).

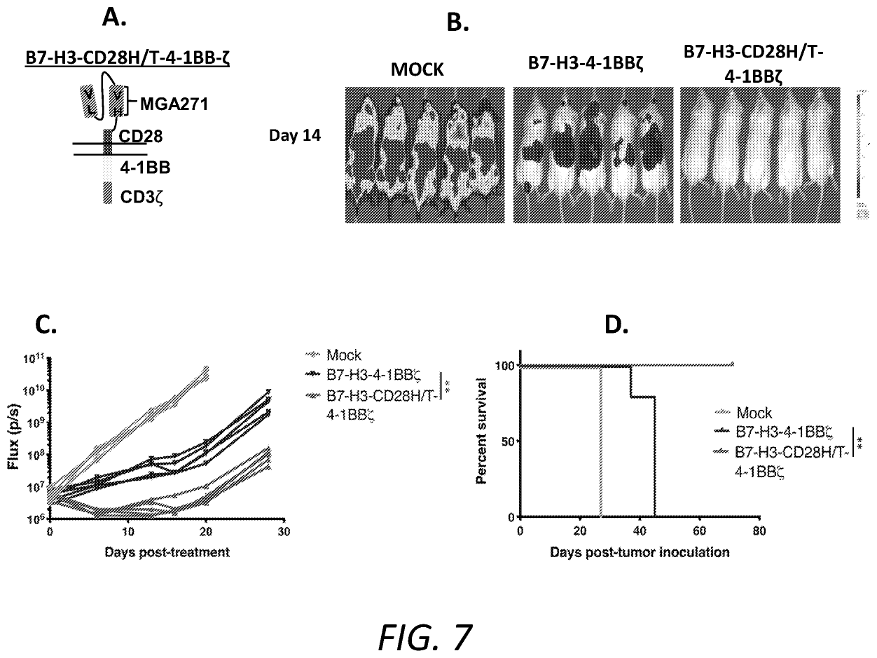

FIGS. 7A-7D schematically summarize the results of experiments performed to assess functionality of CARs targeting B7-H3 in a variety of tumor models and CAR architectures. FIG. 7A Schema of a B7-H3 CAR containing a CD28 hinge-transmembrane region and 4-1BB costimulatory domain (B7-H3-CD28H/T-4-1BBζ). FIG. 7B: One million CHLA255 neuroblastoma cells were engrafted into NSG mice by tail vein injection in a metastatic neuroblastoma model. Six days later, mice were injected with 10 million B7-H3-4-1BB□CAR T cells, B7-H3-CD28H/T-4-1BBζ CAR T cells, or untransduced control T cells (MOCK). Tumor progression was measured by bioluminescence photometry and flux values (photons per second) were calculated using Living Image software. Representative bioluminescent images are shown. FIG. 7C: Quantified tumor flux values for individual mice treated as in FIG. 7B. Statistical analysis performed with repeated measures ANOVA. FIG. 7D: Survival curves for mice treated as in FIG. 7B. Statistical analysis performed with the log-rank test. The results presented in FIGS. 7B-7D are representative of two experiments with different T cell donors. For in vitro experiments, error bars represent SD and for in vivo experiments, error bars represent SEM. p<0.05 was considered statistically significant, and p values are denoted with asterisks as follows: p>0.05, not significant, NS; * p<0.05, ** p<0.01, *** p<0.001, and **** p<0.0001.

FIGS. 8A-8C graphically summarizes the results of experiments suggesting that the CD28 hinge domain is responsible for enhancement in CAR T cell efficacy even in the absence of costimulation (in a first generation CAR construct). FIG. 8A: is a schematic of exemplary first generation CD19 CARs with either a CD8 or CD28 hinge-transmembrane region (CD19-CD8H/T-ζ and CD19-CD28H/T-ζ). FIG. 8B: NALM6 clones expressing either 963 or 45,851 molecules of surface CD19 were co-cultured at a 1:1 ratio with either CD19-CD28ζ, CD19-4-1BBζ, CD19-CD28H/T-ζ or CD19-CD8H/T-ζ CAR T cells and tumor cell killing was measured in an Incucyte assay. Representative of three experiments with different T cell donors. Statistical analysis performed with repeated measures ANOVA between CD19-CD28H/T-ζ and CD19-CD8H/T-ζ. FIG. 8C: CD19-CD28ζ, CD19-4-1BBζ, CD19-CD28H/T-ζ, and CD19-CD8H/T-ζ CAR T cells were co-cultured with NALM6 clones expressing various amounts of CD19 for 24 hours and secreted IL-2 was measured in the supernatant by ELISA. Representative of three experiments with different T cell donors. Statistical comparisons performed with the student's t-test (two sided) between CD19-CD28H/T-ζ and CD19-CD8H/T-ζ.

### Example 6

### Assessing Functionality of CD19 CARs with Different Combinations of Hinge Domains and Transmembrane Domains Derived from Either CD28 or CD8α

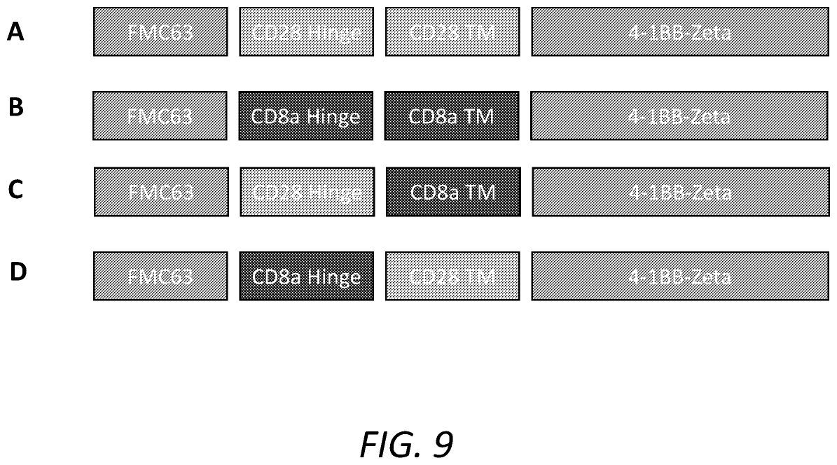

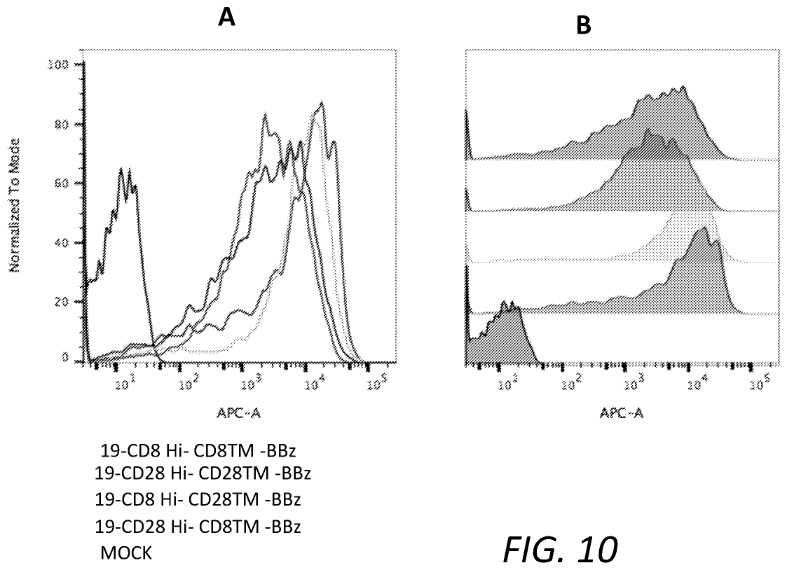

To investigate the functionality of CD19 CARs with different combinations of hinge domains and TMDs, four additional CD19 CARs have been designed and tested (see, e.g., FIGS. 9A-9D). Each of the new CAR design contained an antigen binding moiety derived from the anti-human B cells CD19 antibody (clone FMC63), a costimulatory domain from 4-1BB, a CD3-zeta domain, and different combinations of hinge domains and TMDs derived from either CD28 or CD8α. Expression of the four CD19-targeting CAR designs were then analyzed (FIGS. 10A-10B).

Retroviral vectors encoding CD19 CARs with the indicated structures were synthesized commercially and cloned by standard methods. Viral supernatant was produced in 293GP cells after transient transfection of the retroviral plasmid. Primary human T cells were transduced with viral supernatant after activation with CD3/CD28 beads. It was observed that all of the four CARs described above expressed on the surface of T cells in a similar manner, regardless of the hinge and transmembrane domains. CAR expression was detected with an anti-idiotype antibody that recognized FMC63.

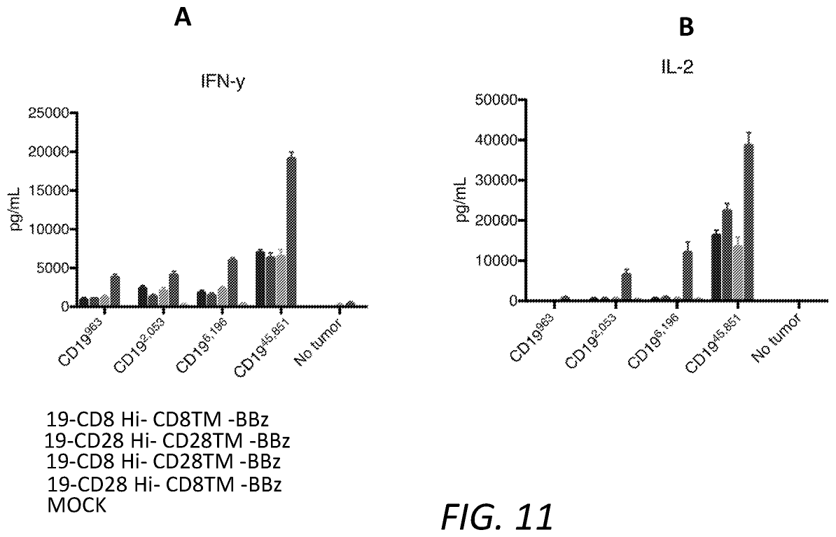

FIGS. 11A-11B summarize the results of experiments suggesting that the CD28 hinge domain is responsible for the enhancement in CAR functionality, and further suggesting that the CD28Hi-CD8TM combination can be a more potent version. In the experiments described at FIG. 11A, CARs with the indicated structure were co-cultured for 24 hours with leukemia lines expressing increasing amounts of CD19 (each clone represents increasing amounts of CD19: z=approximately 1,000 molecules per cell; F=approximately 2,500 per cell; 11=approximately 6,000 molecules per cell; 6=approximately 40,000 molecules per cell) and IFN-γ was measured in the supernatant. As shown in FIG. 11A, CD19 CARs containing a 4-1BB costimulatory domain demonstrated enhanced recognition of low antigen density only when they contained a CD28 hinge domain.

In the experiments described at FIG. 11B, CARs with the indicated structure were co-cultured for 24 hours with leukemia lines expressing increasing amounts of CD19 (each clone represents increasing amounts of CD19: z=approximately 1,000 molecules per cell; F=approximately 2,500 per cell; 11=approximately 6,000 molecules per cell; 6=approximately 40,000 molecules per cell) and IL-2 was measured in the supernatant. CD19 CARs containing a 4-1BB costimulatory domain demonstrated enhanced recognition of low antigen density only when they contained a CD28 hinge domain.

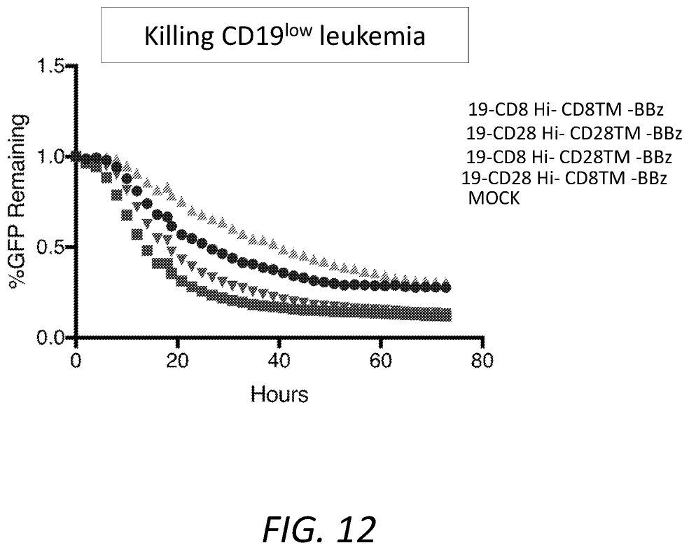

FIG. 12 summarizes the results of experiments suggesting that the CD28 hinge domain is responsible for the enhancement in cell-killing efficacy of low antigen expressing cells. In these experiments, the CD19 CARs with the indicated structures were co-cultured with NALM6 cells expressing very low levels of CD19 (approximately 1000 molecules per cell) and tumor cells remaining were measured over time in an Incucyte by measuring GFP (the NALM6 cells express GFP).

### Example 7

### CD28 Hinge Domain Enhances CAR Activity

This Example describes experiments performed to demonstrate that the CD28 Hinge-TMD results in more efficient receptor clustering, T cell activation, and tumor cell killing, especially at lower target density.

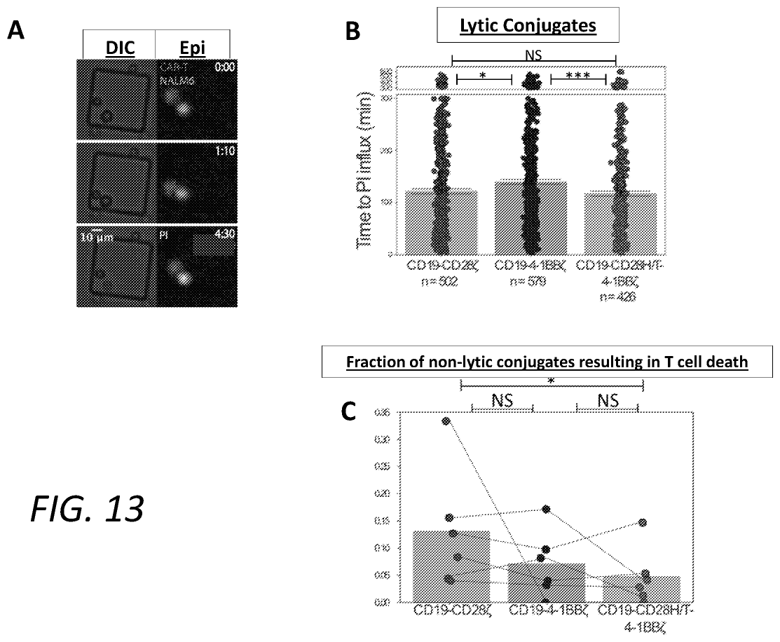

As summarized in FIGS. 13A-13B, CAR T cells and NALM6 cells were seeded at low density on a microwell plate and scanned for wells containing one tumor cell and one CAR T cell. Experiment was performed 6 times across two different T cell donors. As shown in FIG. 13A, a representative well from the single-cell microwell killing experiment is shown. CAR T cells and NALM6 leukemia cells were distinguished by CellTrace Far Red (false-colored magenta) and GFP (false-colored cyan) labels, respectively. Cell death was determined by influx of cell-impermeable propidium iodide dye (PI, false-colored yellow). Lytic conjugates were defined as events where one T cell and one NALM6 cell remained within a threshold distance, and the NALM6 cell died (took up PI). Nonlytic conjugates represent conjugates where the T cell and tumor cell interact but the NALM6 cell did not die (did not take up PI). DIC: Differential interference contrast and Epi: epifluorescence. As shown in FIG. 13B, time from T cell/tumor cell interaction to PI influx was measured in wells containing one tumor cell and one T cell per CAR construct. Pooled data from all 6 experiments (400-600 wells) is shown. Error bars represent SD. Statistical analysis performed with the student's t-test (two sided). As shown in FIG. 13C, the fraction of nonlytic conjugates (conjugates where the T cell and tumor cell interacted but the NALM6 cell did not die) that resulted in T cell death was measured in each of six experiments. The experimental results described in this Example demonstrate that CD28 Hinge/TM endows CAR T cells with the ability to kill faster after target engagement.

### Example 8

### Assessing Functionality of CD28 Hinge in the Context of CARs Targeting Her2 Antigen

This Example describes experiments performed to assessing functionality of CARs targeting Her2 in human 143b obsteosarcoma cells (Her2^{low}) in a cell-killing assay.

In these experiments, one million 143b osteosarcoma cells were orthotopically implanted in the hind leg of NSG mice. After seven days, mice were treated with 10 million Her2-4-1BBζ CAR T cells, Her2-CD28H/T-4-1BBζ CAR T cells, or untransduced control T cells (MOCK). Leg measurements were obtained twice weekly with digital calibers. Measurements for individual mice are shown. Statistical analysis performed with repeated measures ANOVA. FIG. 6C depicts survival curves for mice treated as in FIG. 6B, where statistical analysis performed with the log-rank test. The results presented in FIGS. 6B-6C are representative of two experiments with different T cell donors (n=5 mice per group. The CD28 Hinge-TM domain endows CARS, including those that recognize Her2, with the ability to kill tumor cells in vivo that would not be killed by traditional CAR architecture).

### Example 9

### Assessing Functionality of CD28 Hinge in the Context of CARs Targeting B7-H3 Antigen

This Example describes experiments performed to demonstrate that a hinge domain derived from CD28 can enhance functionality of CARs targeting B7-H3 antigen.

In these experiments, traditional B7-H3-41BBz CAR T cells (containing a CD8 hinge region) were compared to B7-H3 CAR T cells containing the CD28 hinge domain and 4-1BBz endodomains in a prolonged killing assay against the neuroblastoma tumor line CHLA255 in an Incucyte assay. As shown in FIG. 20A, a B7-H3 CAR containing the CD28 hinge region and a 4-1BB costimulatory domain was generated through standard cloning techniques.

T cells were transduced with either B7-H3-4-1BBζ CAR T cells or B7-H3-CD28H/T-4-1BBζ CARs. These CAR T cells were subsequently co-cultured with the neuroblastoma tumor line CHLA255 (transduced with red fluorescent protein) at a 1:4 effector to tumor ratio and compared in a prolonged killing assay in an Incucyte. In these experiments, one million CHLA255 neuroblastoma cells were engrafted into NSG mice by tail vein injection in a metastatic neuroblastoma model. Six days later, mice were injected with 10 million B7-H3-4-1BBζ CAR T cells, B7-H3-CD28H/T-4-1BBζ CAR T cells, or untransduced control T cells (MOCK). Tumor progression was measured by bioluminescence photometry and flux values (photons per second) were calculated using Living Image software. Representative bioluminescent images are shown. As shown in FIG. 7C, quantified tumor flux values for individual mice treated as in FIG. 7B. Statistical analysis performed with repeated measures ANOVA. As shown in FIG. 7D, survival curves for mice treated as in FIG. 7B. Statistical analysis performed with the log-rank test. The results presented in FIGS. 7B-7D are representative of two experiments with different T cell donors. For in vitro experiments, error bars represent SD and for in vivo experiments, error bars represent SEM. p<0.05 was considered statistically significant, and p values are denoted with asterisks as follows: p>0.05, not significant, NS; * p<0.05, ** p<0.01, *** p<0.001, and **** p<0.0001.

As shown in FIGS. 7B-7D, the B7-H3 CAR T cells containing the CD28 hinge domain and 4-1BB-zeta endodomains eradicated tumor cells while those with the traditional CD8 hinge domain and 4-1BB-zeta endodomains did not, resulting in enhanced survival of mice.

### Example 10

### CARs Containing a CD28 Hinge-TM Domain are More Efficient at Clustering in Response to Antigen and Recruiting Proximal Signaling Molecules

This Example describes experiments performed to demonstrate that a hinge-transmembrane domain derived from CD28 enhances CAR T cell immune synapse formation, resulting in improved efficacy, especially in settings in which antigen density are limiting.

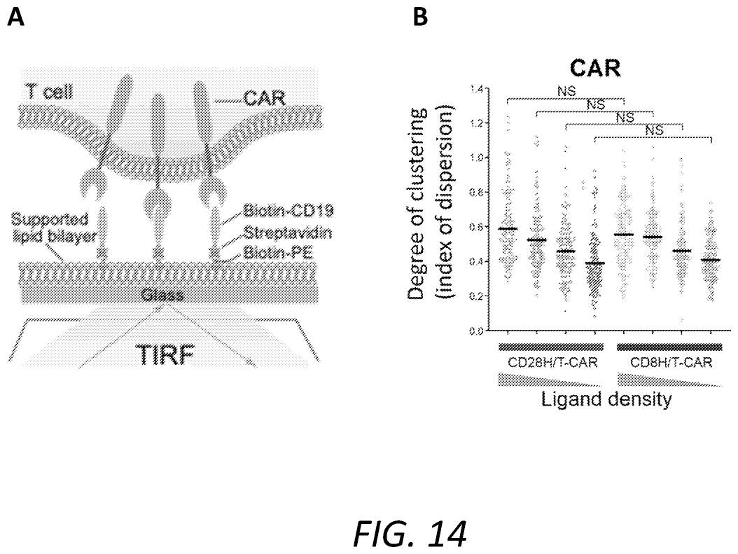

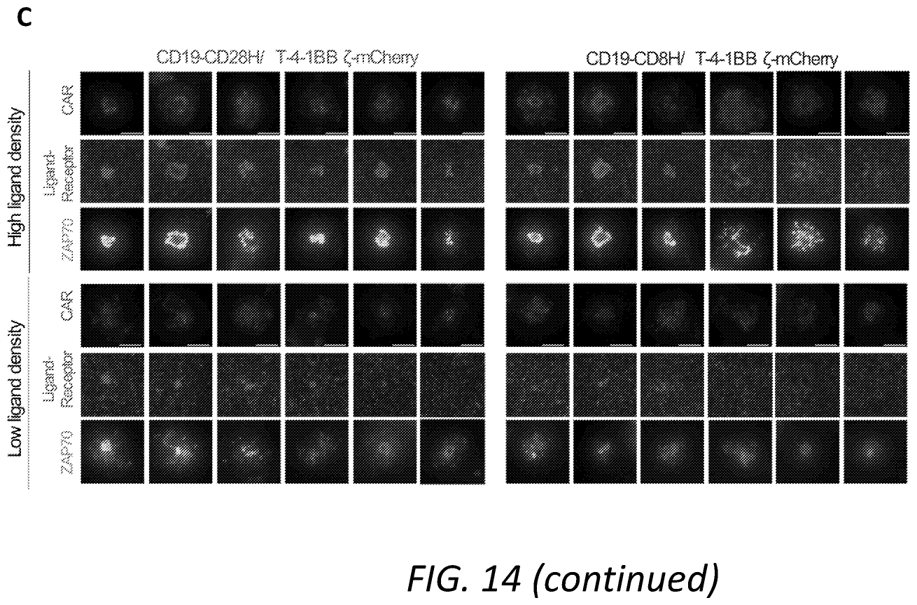

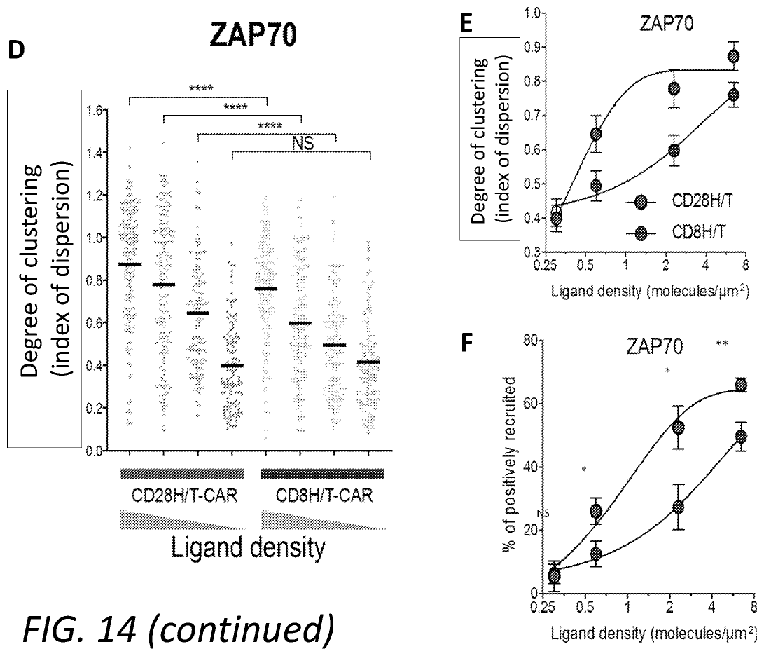

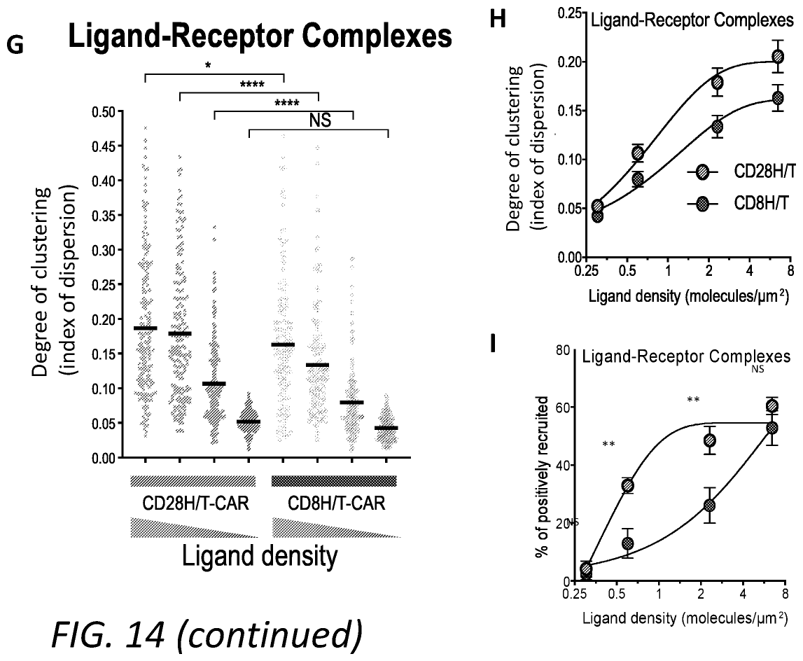

FIGS. 14A-14F schematically summarize the results of additional experiments performed to illustrate that the CD28 Hinge-TMD results in more efficient receptor clustering, T cell activation, and tumor cell killing. A diagram of the imaging-based CAR T cell activation assay is shown in FIG. 14A. To stimulate CD19-CD28H/T-4-1BBζ and CD19-4-1BBζ CAR T cells, CAR T cells were exposed to a planar supported lipid bilayer (SLB) functionalized with a freely diffusing CD19 proteins coupled by a biotin-streptavidin-biotin bridge. Ligand-receptor engagement leads to the reorganization of ligand-bound receptors into microclusters that recruit the tyrosine kinase ZAP70 (fused to GFP, not shown in this diagram) from the cytosol to the plasma membrane, and drive the centripetal translocation of the microclusters from the periphery to the cell center. These events are visualized by TIRF microscopy (fluorescence: CAR-mCherry, ZAP70-GFP, Streptavidin-Alexa647). Ligand density in the planar supported lipid bilayer is controlled through the concentration of Biotin-PE containing small unilamellar vesicles (SUVs). To assess the level of recruitment/degree of clustering across cells that display a range of expression levels, index of dispersion (i.e., normalized variance, which equals the standard deviation divided by the mean of the fluorescence intensity of each cell, see methods for details) was used. As shown in FIG. 14B is the degree of clustering (index of dispersion) for CAR molecules recruited to the immune synapse for each CAR construct at different CD19 densities in the experiment in FIGS. 14C-14I. FIG. 14C show representative images of single CD19-CD28H/T-4-1BBζ-mCherry (left panels) and CD19-CD8H/T-4-1BBζ-mCherry (right panels) CAR T cells transduced with ZAP70-GFP activated on planar supported lipid bilayer containing high (˜6.0 molecule/μm2; top panel) and low (˜0.6 molecule/μm2; bottom panel) concentrations of CD19. FIG. 14D: Degree of clustering (index of dispersion) for ZAP70-GFP recruited to the immune synapse for each CAR construct at four different CD19 densities. FIG. 14E: Pooled ZAP70 degree of clustering (index of dispersion) data from FIG. 14D plotted as a dose response curve for ligand density. FIG. 14F shows percentage of cells activated (ZAP70 recruitment above a threshold) plotted as a dose response curve for ligand density. FIG. 14G shows the degree of clustering (index of dispersion) for ligand-receptor complexes recruited to the immune synapse for each CAR construct at four different CD19 densities. FIG. 14H shows pooled ligand-receptor complex degree of clustering (index of dispersion) data from (h) plotted as a dose response curve for ligand density. FIG. 14I shows percentage of cells recruiting ligand-receptor complexes (above a threshold) plotted as a dose response curve for ligand density. The results presented in FIGS. 14A-14I (shown as mean±SD) are representative from one experiment of two performed with different T cell donors. n>100 per condition. Statistical analysis performed with the two-tailed t-test. p<0.05 was considered statistically significant, and p values are denoted with asterisks as follows: p>0.05, not significant, NS; * p<0.05, ** p<0.01, *** p<0.001, and **** p<0.0001. Data are representative from one experiment with two with different T cell donors. n>100 per condition. Statistical analysis performed with the student's t-test.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A chimeric polypeptide comprising:
a first polypeptide segment comprising an extracellular domain (ECD) capable of binding an antigen;
a second polypeptide segment comprising a hinge domain derived from CD28;
a third polypeptide segment comprising a transmembrane domain (TMD); and
optionally a fourth polypeptide segment comprising an intracellular signaling domain (ICD) comprising one or more costimulatory domains, wherein the one or more costimulatory domains is not from CD28.

2. The chimeric polypeptide of claim 1, wherein the ICD further comprises a CD3ζ ICD.

3. The chimeric polypeptide of any one of claims 1 to 2, wherein the chimeric polypeptide is a chimeric antigen receptor (CAR).

4. The chimeric polypeptide of any one of claims 1 to 3, wherein the antigen is a tumor associated-antigen or a tumor-specific antigen.

5. The chimeric polypeptide of any one of claims 1 to 4, wherein the antigen selected from the group consisting of Glypican 2 (GPC2), IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD19, CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, IgG Kappa light chain, IgA Lamba light chain, CD16/FcγRIII, CD64, FITC, CD27, CD30, CD70, GD2 (ganglioside G2), EGFRvIII (epidermal growth factor variant III), EGFR and isovariants thereof, TEM-8, sperm protein 17 (Sp17), mesothelin, PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3(CD61), galactin, K-Ras (V-Ki-ras2 Kirsten rat sarcoma viral oncogene), and Ral-B.

6. The chimeric polypeptide of any one of claims 1 to 5, wherein the antigen is expressed at low density.

7. The chimeric polypeptide of any one of claims 1 to 6, wherein the antigen is Glypican 2 (GPC2), human epidermal growth factor receptor 2 (Her2/neu), CD276 (B7-H3), or an IL-13-receptor alpha.

8. The chimeric polypeptide of any one of claims 1 to 7, wherein the costimulatory domain is selected from the group consisting of a costimulatory 4-1BB (CD137) polypeptide sequence, a costimulatory CD27 polypeptide sequence, a costimulatory OX40 (CD134) polypeptide sequence, a costimulatory inducible T-cell costimulatory (ICOS) polypeptide sequence, and a CD2 costimulatory domain.

9. The chimeric polypeptide of any one of claims 1 to 8, wherein the costimulatory domains comprises a costimulatory 4-1BB (CD137) polypeptide sequence.

10. The chimeric polypeptide of any one of claims 1 to 9, wherein the TMD is derived from a CD28 TMD, a CD8α TMD, a CD3 TMD, a CD4 TMD, a CTLA4 TMD, and a PD-1 TMD.

11. The chimeric polypeptide of any one of claims 1 to 10, wherein the chimeric polypeptide comprises, in N-terminal to C-terminal direction:
an ECD capable of binding CD19 antigen;
a hinge domain derived from CD28;
a TMD derived from CD8, CD28, CD3, CD4, CTLA4, or PD-1;
an ICD comprising a costimulatory domain from 4-1BB; and
a CD3ζ domain.

12. The chimeric polypeptide of claim 11, wherein the TMD is derived from CD8.

13. The chimeric polypeptide of any one of claims 1 to 10, wherein the chimeric polypeptide comprises, in N-terminal to C-terminal direction:
an ECD capable of binding CD19 antigen;
a hinge domain derived from CD28;
a TMD derived from CD8; and
a CD3ζ domain.

14. The chimeric polypeptide of any one of claims 1 to 10, wherein the chimeric polypeptide comprises, in N-terminal to C-terminal direction:
an ECD capable of binding HER2 antigen;
a hinge domain derived from CD28;
a TMD derived from CD8, CD28, CD3, CD4, CTLA4, or PD-1;
an ICD comprising a costimulatory domain from 4-1BB; and
a CD3ζ domain.

15. The chimeric polypeptide of any one of claims 1 to 10, wherein the chimeric polypeptide comprises, in N-terminal to C-terminal direction:
an ECD capable of binding GPC2 antigen;
a hinge domain from CD28;
a TMD from CD8, CD28, CD3, CD4, CTLA4, or PD-1;
an ICD comprising a costimulatory domain from 4-1BB; and
a CD3ζ domain.

16. The chimeric polypeptide of any one of claims 1 to 10, wherein the chimeric polypeptide comprises, in N-terminal to C-terminal direction:
an ECD capable of binding B7-H3 antigen;
a hinge domain from CD28;
a TMD from CD8, CD28, CD3, CD4, CTLA4, or PD-1;
an ICD comprising a costimulatory domain from 4-1BB; and
a CD3ζ domain.

17. The chimeric polypeptide of any one of claims 1 to 16, wherein the chimeric polypeptide an amino acid sequence having at least 80% sequence identity to an amino acid sequence selected from the group consisting of SEQ ID NO: 13, SEQ ID NO: 27, SEQ ID NO: 39, SEQ ID NO: 53, and SEQ ID NO: 67.

18. A recombinant nucleic acid molecule comprising a nucleic acid sequence that encodes a chimeric polypeptide according to of any one of claims 1 to 17.

19. The recombinant nucleic acid molecule of claim 18, wherein the nucleic acid sequence has at least 80% sequence identity to a nucleic acid sequence selected from the group consisting of SEQ ID NO: 14, SEQ ID NO: 28, SEQ ID NO: 40, SEQ ID NO: 54, and SEQ ID NO: 68.

20. The recombinant nucleic acid molecule of any one of claims 18 to 19, wherein the recombinant nucleic acid molecule is operably linked to a heterologous nucleic acid sequence.

21. The recombinant nucleic acid molecule of any one of claims 18 to 20, wherein the recombinant nucleic acid molecule is further defined as an expression cassette in a vector.

22. The nucleic acid molecule of claim 21, wherein the vector is a plasmid vector or a viral vector.

23. The nucleic acid molecule of claim 22, wherein the viral vector is derived from a lentivirus, an adeno virus, an adeno-associated virus, a baculovirus, or a retrovirus.

24. A recombinant cell comprising:
a chimeric polypeptide according to any one of claims 1 to 17; and/or
a nucleic acid molecule according to any one of claims 18 to 23;

25. The recombinant cell of claim 24, wherein the recombinant cell is a eukaryotic cell.

26. The recombinant cell of any one of claims 24 to 25, wherein the recombinant cell is an immune system cell.

27. The recombinant cell of claim 26, wherein the immune system cell is a T lymphocyte.

28. A method for making a recombinant cell, comprising:
a) providing a host cell capable of protein expression; and
b) transducing the provided host cell with a recombinant nucleic acid according to any one of claims 18 to 23 to produce a recombinant cell.

29. A recombinant cell produced by a method according to claim 28.

30. A cell culture comprising at least one recombinant cell according to any one of claims 24 to 27 and a culture medium.

31. A pharmaceutical composition comprising a pharmaceutically acceptable carrier and:
a) a chimeric polypeptide according to any one of claims 1 to 17;
b) a nucleic acid molecule according to any one of claims 18 to 23; and/or
c) a recombinant cell according to any one of claims 24-27 and 29.

32. The pharmaceutical composition of claim 31, wherein the composition comprises a recombinant nucleic acid according to any one of claims 18 to 23, a pharmaceutically acceptable carrier.

33. The pharmaceutical composition of claim 32, wherein the recombinant nucleic acid is encapsulated in a viral capsid or a lipid nanoparticle.

34. The pharmaceutical composition of claim 31, wherein the composition comprises a recombinant cell according to any one of claims 24-27 and 29, a pharmaceutically acceptable carrier.

35. A method for preventing and/or treating a condition in a subject in need thereof, comprising administering to the subject a composition comprising:
a) a chimeric polypeptide according to any one of claims 1 to 17;
b) a nucleic acid molecule according to any one of claims 18 to 23;
c) a recombinant cell according to any one of claims 24-27 and 29; and/or
d) a pharmaceutical composition according to any one of claims 31 to 34.

36. The method of claim 35, wherein the condition is a cancer.

37. The method of claim 36, wherein the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

38. The method of any one of claims 35 to 37, wherein the administered composition confers increased production of interferon gamma (IFNγ) and/or interleukin-2 (IL-2) in the subject.

39. The method of any one of claims 35 to 38, wherein the administered composition inhibits tumor growth or metastasis of the cancer in the subject.

40. The method of any one of claims 35 to 39, wherein the composition is administered to the subject individually as a first therapy or in combination with a second therapy.

41. The method of claim 40, wherein the second therapy is selected from the group consisting of chemotherapy, radiotherapy, immunotherapy, hormonal therapy, toxin therapy, and surgery.

42. The method of any one of claims 40 to 41, wherein the first therapy and the second therapy are administered concomitantly.

43. The method of any one of claims 40 to 42, wherein the first therapy is administered at the same time as the second therapy.

44. The method of any one of claims 40 to 41, wherein the first therapy and the second therapy are administered sequentially.

45. The method of claim 44, wherein the first therapy is administered before the second therapy.

46. The method of claim 44, wherein the first therapy is administered after the second therapy.

47. The method of any one of claims 40 to 41, wherein the first therapy is administered before and/or after the second therapy.

48. The method of any one of claims 40 to 41, wherein the first therapy and the second therapy are administered in rotation.

49. The method of any one of claims 40 to 41, wherein the first therapy and the second therapy are administered together in a single formulation.

50. A kit for the diagnosis, prevention, and/or treatment a condition in a subject in need thereof, the kit comprising:
a) a chimeric polypeptide according to any one of claims 1 to 17;
b) a nucleic acid molecule according to any one of claims 18 to 23;
c) a recombinant cell according to any one of claims 24-27 and 29; and/or
d) a pharmaceutically composition according to any one of claims 31 to 34.


SEQUENCE LISTING (68 sequences)

SEQ ID NO: 1 - protein, 22 aa, Artificial sequence
  Synthetic polypeptide
  MISC_FEATURE: signal peptide
MLLLVTSLLLCELPHPAFLLIP

SEQ ID NO: 2 - DNA, 66 bp, Artificial sequence
  Synthetic polypeptide
  misc_feature: encodes the polypeptide of SEQ ID NO 1
atgcttctcctggtgacaagccttctgctctgtgagttaccacacccagcattcctcctgatccca

SEQ ID NO: 3 - protein, 245 aa, Artificial sequence
  Synthetic polypeptide
  MISC_FEATURE: Anti-CD19 ScFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 735 aa, Artificial sequence
  Synthetic polypeptide
  MISC_FEATURE: encodes the polypeptide of SEQ ID NO 3
GACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACTAAGTTGGAAATAACAGGCTCCACCTCTGGATCCGGCAAGCCCGGATCTGGCGAGGGATCCACCAAGGGCGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGTCAAGGAACCTCAGTCACCGTCTCCTCA

SEQ ID NO: 5 - protein, 39 aa, Artificial sequence
  Synthetic polypeptide
  MISC_FEATURE: CD28 Hinge Domain
IEVMYPPPYLDNEKSNGTIIHVKGKHLCPSPLFPGPSKP

SEQ ID NO: 6 - DNA, 117 bp, Artificial sequence
  Synthetic polypeptide
  misc_feature: encodes the polypeptide of SEQ ID NO 5
attgaagttatgtatcctcctccttacctagacaatgagaagagcaatggaaccattatccatgtgaaagggaaacacctttgtccaagtcccctatttcccggaccttctaagccc

SEQ ID NO: 7 - protein, 27 aa, Artificial sequence
  Synthetic polypeptide
  MISC_FEATURE: CD28 Transmembrane domain
FWVLVVVGGVLACYSLLVTVAFIIFWV

SEQ ID NO: 8 - DNA, 71 bp, Artificial sequence
  Synthetic polypeptide
  misc_feature: encodes the polypeptide of SEQ ID NO 7
atctacatctgggcgcccttggccgggacttgtggggtccttctcctgtcactggttatcaccctttactg

SEQ ID NO: 9 - protein, 42 aa, Artificial sequence
  Synthetic polypeptide
  MISC_FEATURE: 4-1BB
KRGRKKLLYIFKQPFMRPVQTTQEEDGCSCRFPEEEEGGCEL

SEQ ID NO: 10 - DNA, 126 bp, Artificial sequence
  Synthetic polypeptide
  misc_feature: encodes the polypeptide of SEQ ID NO 9
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 11 - protein, 112 aa, Artificial sequence
  Synthetic polypeptide
  MISC_FEATURE: CD3-zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 339 bp, Artificial sequence
  Synthetic polypeptide
  misc_feature: encodes the polypeptide of SEQ ID NO 11
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 13 - protein, 490 aa, Artificial sequence
  Synthetic polypeptide
  MISC_FEATURE: signal peptide-anti CD19 ScFv-CD28hinge-CD28TM- 41BB-zeta
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 1473 bp, Artificial sequence
  Synthetic polypeptide
  misc_feature: encodes the polypeptide of SEQ ID NO 13
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 15 - protein, 22 aa, Artificial sequence
  Synthetic polypeptide
  MISC_FEATURE: signal Peptide
MLLLVTSLLLCELPHPAFLLIP

SEQ ID NO: 16 - DNA, 66 bp, Artificial sequence; identical to SEQ ID NO: 2
  Synthetic polypeptide
  misc_feature: encodes the polypeptide of SEQ ID NO 15

SEQ ID NO: 17 - protein, 245 aa, Artificial sequence; identical to SEQ ID NO: 3
  Synthetic polypeptide
  MISC_FEATURE: Anti-CD19 ScFv

SEQ ID NO: 18 - DNA, 735 bp, Artificial sequence
  Synthetic polypeptide
  misc_feature: encodes the polypeptide of SEQ ID NO 17
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 19 - protein, 39 aa, Artificial sequence; identical to SEQ ID NO: 5
  Synthetic polypeptide
  MISC_FEATURE: CD28 Hinge Domain

SEQ ID NO: 20 - DNA, 117 bp, Artificial sequence; identical to SEQ ID NO: 6
  Synthetic polypeptide
  misc_feature: encodes the polypeptide of SEQ ID NO 19

SEQ ID NO: 21 - protein, 24 aa, Artificial sequence
  Synthetic polypeptide
  MISC_FEATURE: CD8 Transmembrane domain
IYIWAPLAGTCGVLLLSLVITLYC

SEQ ID NO: 22 - DNA, 71 bp, Artificial sequence; identical to SEQ ID NO: 8
  Synthetic polypeptide
  misc_feature: encodes the polypeptide of SEQ ID NO 21

SEQ ID NO: 23 - protein, 42 aa, Artificial sequence; identical to SEQ ID NO: 9
  Synthetic polypeptide
  MISC_FEATURE: 4-1BB

SEQ ID NO: 24 - DNA, 126 bp, Artificial sequence; identical to SEQ ID NO: 10
  Synthetic polypeptide
  misc_feature: encodes the polypeptide of SEQ ID NO 23

SEQ ID NO: 25 - protein, 112 aa, Artificial sequence; identical to SEQ ID NO: 11
  Synthetic polypeptide
  MISC_FEATURE: CD3-zeta

SEQ ID NO: 26 - DNA, 339 bp, Artificial sequence; identical to SEQ ID NO: 12
  Synthetic polypeptide
  misc_feature: encodes the polypeptide of SEQ ID NO 25

SEQ ID NO: 27 - protein, 487 aa, Artificial sequence
  Synthetic polypeptide
  MISC_FEATURE: signal peptide-anti CD19 ScFv-CD28hinge-CD8TM- 41BB-zeta
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 1463 bp, Artificial sequence
  Synthetic polypeptide
  misc_feature: encodes the polypeptide of SEQ ID NO 27
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccatctacatctgggcgcccttggccgggacttgtggggtccttctcctgtcactggttatcaccctttactgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 29 - protein, 22 aa, Artificial sequence
  Synthetic polypeptide
  MISC_FEATURE: Signal Peptide
MLLLVTSLLLCELPHPAFLLIP

SEQ ID NO: 30 - DNA, 66 bp, Artificial sequence; identical to SEQ ID NO: 2
  Synthetic polypeptide
  misc_feature: encodes the polypeptide of SEQ ID NO 29

SEQ ID NO: 31 - protein, 245 aa, Artificial sequence; identical to SEQ ID NO: 3
  Synthetic polypeptide
  MISC_FEATURE: Anti-CD19 ScFv

SEQ ID NO: 32 - DNA, 735 bp, Artificial sequence; identical to SEQ ID NO: 18
  Synthetic polypeptide
  misc_feature: encodes the polypeptide of SEQ ID NO 31

SEQ ID NO: 33 - protein, 39 aa, Artificial sequence; identical to SEQ ID NO: 5
  Synthetic polypeptide
  MISC_FEATURE: CD28 Hinge domain

SEQ ID NO: 34 - DNA, 117 bp, Artificial sequence; identical to SEQ ID NO: 6
  Synthetic polypeptide
  misc_feature: encodes the polypeptide of SEQ ID NO 33

SEQ ID NO: 35 - protein, 27 aa, Artificial sequence
  Synthetic polypeptide
  MISC_FEATURE: CD28 Transmembrane domain
FWVLVVVGGVLACYSLLVTVAFIIFWV

SEQ ID NO: 36 - DNA, 81 bp, Artificial sequence
  Synthetic polypeptide
  misc_feature: encodes the polypeptide of SEQ ID NO 35
ttttgggtgctggtggtggttgggggagtcctggcttgctatagcttgctagtaacagtggcctttattattttctgggtg

SEQ ID NO: 37 - protein, 112 aa, Artificial sequence; identical to SEQ ID NO: 11
  Synthetic polypeptide
  MISC_FEATURE: CD3-zeta

SEQ ID NO: 38 - DNA, 339 bp, Artificial sequence; identical to SEQ ID NO: 12
  Synthetic polypeptide
  misc_feature: encodes the polypeptide of SEQ ID NO 37

SEQ ID NO: 39 - protein, 448 aa, Artificial sequence
  Synthetic polypeptide
  MISC_FEATURE: signal-peptide-anti CD19 ScFv-CD28hinge-CD28TM- zeta
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1347 bp, Artificial sequence
  Synthetic polypeptide
  misc_feature: encodes the polypeptide of SEQ ID NO 39
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 41 - protein, 21 aa, Artificial sequence
  Synthetic polypeptide
  MISC_FEATURE: Signal Peptide
MARSVTLVFLVLVSLTGLYAA

SEQ ID NO: 42 - DNA, 63 bp, Artificial sequence
  Synthetic polypeptide
  misc_feature: encodes the polypeptide of SEQ ID NO 41
atggctcgctcggtgaccctggtctttctggtgcttgtctcactgaccggtttgtatgctgct

SEQ ID NO: 43 - protein, 243 aa, Artificial sequence
  Synthetic polypeptide
  MISC_FEATURE: Anti-Her2 ScFv
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 44 - DNA, 729 bp, Artificial sequence
  Synthetic polypeptide
  misc_feature: encodes the polypeptide of SEQ ID NO 43
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaagggtctacatctggatctgggaagccgggttctggtgagggttctggtgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcg

SEQ ID NO: 45 - protein, 39 aa, Artificial sequence; identical to SEQ ID NO: 5
  Synthetic polypeptide
  MISC_FEATURE: CD28 Hinge

SEQ ID NO: 46 - DNA, 117 bp, Artificial sequence; identical to SEQ ID NO: 6
  Synthetic polypeptide
  misc_feature: encodes the polypeptide of SEQ ID NO 45

SEQ ID NO: 47 - protein, 27 aa, Artificial sequence
  Synthetic polypeptide
  MISC_FEATURE: CD28 Transmembrane domain
FWVLVVVGGVLACYSLLVTVAFIIFWV

SEQ ID NO: 48 - DNA, 81 bp, Artificial sequence; identical to SEQ ID NO: 36
  Synthetic polypeptide
  misc_feature: encodes the polypeptide of SEQ ID NO 47

SEQ ID NO: 49 - protein, 42 aa, Artificial sequence; identical to SEQ ID NO: 9
  Synthetic polypeptide
  MISC_FEATURE: 4-1BB

SEQ ID NO: 50 - DNA, 126 bp, Artificial sequence; identical to SEQ ID NO: 10
  Synthetic polypeptide
  misc_feature: encodes the polypeptide of SEQ ID NO 49

SEQ ID NO: 51 - protein, 112 aa, Artificial sequence; identical to SEQ ID NO: 11
  Synthetic polypeptide
  MISC_FEATURE: CD3-zeta

SEQ ID NO: 52 - DNA, 339 bp, Artificial sequence; identical to SEQ ID NO: 12
  Synthetic polypeptide
  misc_feature: encodes the polypeptide of SEQ ID NO 51

SEQ ID NO: 53 - protein, 487 aa, Artificial sequence
  Synthetic polypeptide
  MISC_FEATURE: signal peptide-anti Her2 ScFv-CD28Hinge-CD28TM- 41BB-zeta
MARSVTLVFLVLVSLTGLYAADIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1464 bp, Artificial sequence
  Synthetic polypeptide
  misc_feature: encodes the polypeptide of SEQ ID NO 53
atggctcgctcggtgaccctggtctttctggtgcttgtctcactgaccggtttgtatgctgctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaagggtctacatctggatctgggaagccgggttctggtgagggttctggtgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 55 - protein, 24 aa, Artificial sequence
  Synthetic polypeptide
  MISC_FEATURE: Signal Peptide
MVATLLVTSLLLCELPHPAFLLIP

SEQ ID NO: 56 - DNA, 72 bp, Artificial sequence
  Synthetic polypeptide
  misc_feature: encodes the polypeptide of SEQ ID NO 55
atggttgccaccctgctcgtgacaagcctgctgctgtgcgagctgccccaccctgcctttctgctgatcccc

SEQ ID NO: 57 - protein, 249 aa, Artificial sequence
  Synthetic polypeptide
  MISC_FEATURE: Anti-B7-H3 ScFv
DTEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKAAA

SEQ ID NO: 58 - DNA, 747 bp, Artificial sequence
  Synthetic polypeptide
  misc_feature: encodes the polypeptide of SEQ ID NO 57
gataccgaggtgcagctggtggaatctggcggcggactggtgcagcctggcggatctctgagactgagctgtgccgccagcggcttcaccttcagcagcttcggaatgcactgggtgcgccaggcccctggcaaaggactggaatgggtggcctacatcagcagcgacagcagcgccatctactacgccgacaccgtgaagggccggttcaccatctcccgggacaacgccaagaacagcctgtacctgcagatgaactccctgcgggacgaggacaccgccgtgtactattgcggcagaggcagagagaacatctattacggcagcagactggactactggggccagggcacaaccgtgacagtgtctagcggaggcggaggatcaggcggcggaggaagtggcggagggggatctgatatccagctgacccagagccccagcttcctgagcgcctctgtgggcgacagagtgaccatcacatgcaaggccagccagaacgtggacaccaacgtggcctggtatcagcagaagcccggcaaggcccctaaggccctgatctacagcgccagctaccggtacagcggcgtgcccagcagattttctggcagcggctccggcaccgacttcaccctgacaatcagcagcctgcagcccgaggacttcgccacctactactgccagcagtacaacaactaccctttcaccttcggccaggggaccaagctggaaatcaaagcggccgca

SEQ ID NO: 59 - protein, 39 aa, Artificial sequence; identical to SEQ ID NO: 5
  Synthetic polypeptide
  MISC_FEATURE: CD28 Hinge

SEQ ID NO: 60 - DNA, 117 bp, Artificial sequence; identical to SEQ ID NO: 6
  Synthetic polypeptide
  misc_feature: encodes the polypeptide of SEQ ID NO 59

SEQ ID NO: 61 - protein, 27 aa, Artificial sequence
  Synthetic polypeptide
  MISC_FEATURE: CD28 Transmembrane domain
FWVLVVVGGVLACYSLLVTVAFIIFWV

SEQ ID NO: 62 - DNA, 81 bp, Artificial sequence; identical to SEQ ID NO: 36
  Synthetic polypeptide
  misc_feature: encodes the polypeptide of SEQ ID NO 61

SEQ ID NO: 63 - protein, 42 aa, Artificial sequence; identical to SEQ ID NO: 9
  Synthetic polypeptide
  MISC_FEATURE: 4-1BB

SEQ ID NO: 64 - DNA, 126 bp, Artificial sequence; identical to SEQ ID NO: 10
  Synthetic polypeptide
  misc_feature: encodes the polypeptide of SEQ ID NO 63

SEQ ID NO: 65 - protein, 112 aa, Artificial sequence; identical to SEQ ID NO: 11
  Synthetic polypeptide
  MISC_FEATURE: CD3-zeta

SEQ ID NO: 66 - DNA, 339 bp, Artificial sequence; identical to SEQ ID NO: 12
  Synthetic polypeptide
  misc_feature: encodes the polypeptide of SEQ ID NO 65

SEQ ID NO: 67 - protein, 496 aa, Artificial sequence
  Synthetic polypeptide
  MISC_FEATURE: signal peptide-anti B7-H3 ScFv-CD28Hinge- CD28TM-41BB-zeta
MVATLLVTSLLLCELPHPAFLLIPDTEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKAAAAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1491 bp, Artificial sequence
  Synthetic polypeptide
  misc_feature: encodes the polypeptide of SEQ ID NO 67
atggttgccaccctgctcgtgacaagcctgctgctgtgcgagctgccccaccctgcctttctgctgatccccgataccgaggtgcagctggtggaatctggcggcggactggtgcagcctggcggatctctgagactgagctgtgccgccagcggcttcaccttcagcagcttcggaatgcactgggtgcgccaggcccctggcaaaggactggaatgggtggcctacatcagcagcgacagcagcgccatctactacgccgacaccgtgaagggccggttcaccatctcccgggacaacgccaagaacagcctgtacctgcagatgaactccctgcgggacgaggacaccgccgtgtactattgcggcagaggcagagagaacatctattacggcagcagactggactactggggccagggcacaaccgtgacagtgtctagcggaggcggaggatcaggcggcggaggaagtggcggagggggatctgatatccagctgacccagagccccagcttcctgagcgcctctgtgggcgacagagtgaccatcacatgcaaggccagccagaacgtggacaccaacgtggcctggtatcagcagaagcccggcaaggcccctaaggccctgatctacagcgccagctaccggtacagcggcgtgcccagcagattttctggcagcggctccggcaccgacttcaccctgacaatcagcagcctgcagcccgaggacttcgccacctactactgccagcagtacaacaactaccctttcaccttcggccaggggaccaagctggaaatcaaagcggccgcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa